(19) United States
(12) Patent Application Publication
(10) Pub. No.: US 2007/0112361 A1
(43) Pub. Date: May 17, 2007

(54) SURGICAL REPAIR SYSTEMS AND METHODS OF USING THE SAME

(76) Inventors: Steven M. Schonholz, West Brookfield, MA (US); Richard Cayer Jr., Charlton, MA (US); Brett T. Haarala, Framingham, MA (US)

Correspondence Address: CANTOR COLBURN, LLP, 55 GRIFFIN ROAD SOUTH, BLOOMFIELD, CT, 06002, US

(21) Appl. No.: 11/556,893

(22) Filed: Nov. 6, 2006

Related U.S. Application Data
(60) Provisional application No. 60/734,191, filed on Nov. 7, 2005.

(51) Int. Cl. A61B 17/08

(52) U.S. Cl. 606/151

(57) ABSTRACT

Disclosed herein are surgical repair systems and methods of using the same. In one embodiment, a repair system comprises: a positioning device comprising a shaft, a tip section, and an optional depth stop. The tip section comprises a material receiving area configured to receive a fold of a folded material. The tip section is configured to enable wrapping of the folded material around the tip section. The depth stop is configured to inhibit the folded material from moving toward the shaft beyond a desired point. In another embodiment, a repair system comprises: a positioning device comprising a shaft and a tip section. The tip section comprises a material receiving area and a retractable extension wire. The tip section is configured to receive material and retain the material until it is deployed at a defect site. The extension wire is configured to extend from the tip section to support the material.

### CROSS-REFERENCE TO RELATED APPLICATION

This application claims priority to U.S. Provisional Application No. 60/734,191, filed Nov. 7, 2005, which is incorporated herein by reference in its entirety.

### BACKGROUND

This disclosure generally relates to laparoscopic surgical repair, and more particularly to repair systems and devices.

Surgical repair of tissues using materials inserted into the body commonly includes repair of a defect in the abdominal wall, or hernia. A hernia can generally be described as a protrusion of an organ or bodily part through connective tissue or through the wall of the cavity in which it is normally enclosed. These abnormalities can be categorized with respect to the anatomic position of the hernia. An inguinal hernia is the most common type of hernia, which describes a hernia of the groin, wherein abdominal contents (e.g., intestine) can protrude from the abdomen through a defect in the inguinal canal. Inguinal hernias can further be described as “indirect” or “direct”. Indirect inguinal hernias are defects within the apex of the inguinal canal, occurring at the internal ring. Direct hernias are defects within the back wall of the inguinal canal, medial to the spermatic cord. Other abdominal hernias include; femoral hernias, which occur below the groin crease, umbilical hernias, which occur at the umbilical cord, ventral hernias, which occur at the midline of the abdomen, and diaphragmatic hernias, which occur high in the abdominal cavity near the chest. Moreover, hernias can also result from a prior incision that has not properly healed and has reopened, which is referred to as incisional hernias.

The conventional herniorrhaphy surgical procedure for umbilical and ventral hernias comprises creating a single incision several inches in length through the abdominal wall and into the abdominal cavity, which can enable the identification of the defect and hernia contents. In inguinal hernia repair, the hernia can be identified from the weakness that comes from the abdominal cavity. If the hernia is reducible, the herniated tissues can be pushed back into the abdominal cavity, and the defect can be fixed by fixedly attaching a prosthetic reinforcing material (e.g., mesh) or by closing the defect primarily utilizing sutures.

As less invasive surgical techniques are advancing in the field of hernia repair., there is a growing need for innovative laparoscopic compatible devices that alleviate shortcomings in the art and provide novel solutions for laparoscopic hernia repair. Disclosed herein are devices and methods for their use that provide such needed innovations.

### BRIEF SUMMARY

Disclosed herein are surgical repair systems and methods of using the same.

In one embodiment, a repair system comprises: a positioning device comprising a shaft, a tip section, and an optional depth stop. The tip section comprises a material receiving area configured to receive a fold of a folded material. The tip section is configured to enable wrapping of the folded material around the tip section. The depth stop is configured to inhibit the folded material from moving toward the shaft beyond a desired point.

In another embodiment, a repair system comprises: a positioning device comprising a shaft and a tip section. The tip section comprises a material receiving area and a retractable extension wire. The tip section is configured to receive material and retain the material until it is deployed at a defect site. The extension wire is configured to extend from the tip section to support the material during deployment.

In one embodiment, a method for operating a repair system comprises: folding a material to form a folded material, inserting the folded material into a material receiving area in a positioning device, inserting the tip section and folded material into a tapered element at a proximal end of an introducer device, wrapping the folded material around the tip section to form a wrapped material passing the wrapped material through the introducer device, unwrapping the wrapped material to form an unwrapped material, positioning the unwrapped material in a desired location, and securing the unwrapped material to a repair site. The introducer device comprises the proximal end for receiving the tip section and a distal end for deploying the material. The positioning device comprises shaft and a tip section. The tip section comprises the material receiving area.

In another embodiment, a method for operating a repair system comprises: folding a material to form a folded material, inserting the folded material into a material receiving area in a positioning device, wrapping the folded material around the tip section, adjacent to a wide end of the conical tip to form a wrapped material, passing the wrapped material through an abdominal wall into an abdominal cavity, unwrapping the wrapped material to form an unwrapped material, positioning the unwrapped material in a desired location, and securing the unwrapped material to a repair site. The positioning device comprises shaft and a tip section. The tip section comprises the material receiving area and a conical tip.

In yet another embodiment, a method for operating a repair system comprises: folding a material to form a folded material, insert a positioning device through an introducer device such that a tip section of the positioning device extends out of a distal end of the introducer device, inserting the folded material into the first material receiving area, inserting the tip section and folded material into a second material receiving area, wrapping the folded material around the tip section to form a wrapped material, introducing the wrapped material to an abdominal cavity, unwrapping the wrapped material to form an unwrapped material, positioning the unwrapped material in a desired location, and securing the unwrapped material to a repair site. The tip section comprises a first material receiving area. The folded material can extend into the first material receiving area up to a stop.

The above described and other features are exemplified by the following figures and detailed description.

### BRIEF DESCRIPTION OF THE DRAWINGS

Referring now to the figures, which are exemplary embodiments, and wherein like elements are numbered alike.

FIG. 1 is an oblique view of an exemplary positioning device.

FIG. 2a is an isometric view of an exemplary material (e.g., hernia mesh) being folded.

FIG. 2b is a partial, isometric view of an exemplary folded material being inserted into the tip section of a positioning device.

FIG. 2c is a partial, isometric view of an exemplary folded material loaded in a tip section of a positioning device.

FIG. 3 is a partial side view of the exemplary wrapping of a folded material.

FIG. 4a is a partial side view of an exemplary insertion of a wrapped material into a trocar cannula.

FIG. 4b is a view of material wrapped in a pitched helical spiral.

FIG. 5a is a partial, isometric view of an exemplary folded material loaded into a positioning device, inserted into a slot of an exemplary wrapping/introducer tube, and rotated.

FIG. 5b is a partial, isometric view of an exemplary wrapped material and positioning device inside the exemplary wrapping/introducer tube.

FIG. 6 is a side view of the exemplary positioning of a folded repair material at a defect site using a material placement system.

FIG. 7a is an oblique view of an exemplary wrapping/insertion device.

FIG. 7b is an oblique view of another exemplary wrapping/insertion device with a slot at the distal end.

FIGS. 7c and 7d are cross-sectional and isometric views, respectively, of another embodiment of a wrapping/introducer device representing a very short (e.g., less than 1 centimeter) or no tubular section.

FIG. 7e is partial, detailed, side view of an exemplary flared material receiving area of the introducer tube in FIG. 7b.

FIG. 8 is an isometric view of an assembly comprised of an exemplary positioning device inserted in an exemplary trocar cannula/wrapping device with a slot for wrapping of material.

FIG. 9 is an isometric view of an exemplary curved positioning device.

FIG. 10 is a cross-sectional side view of an exemplary rotatable tip positioning device.

FIG. 11 is a partial, isometric view of a rotatable tip positioning device comprising a curved distal section.

FIG. 12 a partial, isometric view of an exemplary tip section of an exemplary positioning device comprising a loading pin.

FIG. 13 is a partial, isometric view of the exemplary loading of a material in the end of the positioning device of FIG. 12.

FIG. 14 is a partial, isometric view of the exemplary the folding of a material around the loading pin of the positioning device of FIG 12.

FIG. 15 is a cross-sectional side view of an exemplary retractable pin positioning device.

FIG. 16a is an end view of an exemplary friction pin.

FIG. 16b is an end view of the friction pin of FIG. 16a compressing a folded material.

FIG. 17 is a partial, isometric view of an exemplary loading pin comprising an atraumatic pin.

FIG. 18 is a cross-sectional view of a retractable pin positioning device configured with a depth stop insert.

FIG. 19a is a partial, isometric view of an exemplary material receiving area with a length adjustment ring that provides a depth stop to prevent movement toward the shaft beyond a desired point.

FIG. 19b is a partial, isometric view of an exemplary adjustable-tip positioning device.

FIG. 20 is a partial, isometric view of an exemplary two-projection tip.

FIG. 21 is a cross-sectional view of an exemplary actuating positioning system and introducer tube.

FIG. 22 is a partial side view of another embodiment of a two projection tip.

FIG. 23 is a partial, isometric view of an exemplary three-projection tip configuration.

FIG. 24 is an isometric view of an exemplary introducer/trocar with a material reception area to allow wrapping of material inside the introducer trocar prior to insertion in the body.

FIG. 25 is a cross-sectional illustration of an exemplary material placement system comprising an introducer/trocar with a rolled polymer sheath assembled with a positioning device having a conical tip and loading pin that are configured to allow the insertion into an incision in the body as well as loading with folded material.

FIG. 26 is a side view of an exemplary introducer tube comprising an unrolled polymer sheath.

FIG. 27 is a cross-sectional view of an exemplary steerable positioning device.

FIG. 28 is an isometric view of an exemplary two-projection tip comprising extension wires.

FIG. 29 is an isometric view of an exemplary two-projection tip comprising deployed extension wires.

FIG. 30a is a detailed view of the tip of an exemplary positioning device with a tip configured for insertion in the body illustrating the loading pin and material reception area.

FIG. 30b is an isometric view of the positioner tip of FIG. 30a.

FIG. 31a is a cross-sectional view of another embodiment of a positioner tip with a flexible material support member adjacent to the material reception area.

FIG. 31b is a cross-sectional view of yet another embodiment of a positioner tip with a flexible material enclosing and adjacent to the material reception area.

FIG. 31c is a cross-sectional view of the positioner tip of FIG. 31a loaded with a flexible material.

FIG. 31d is a cross-sectional view of another embodiment of a positioner tip with a flexible material support member adjacent to the material reception area.

FIGS. 32a, 32b, and 32c, are cross-sectional views of alternate positioner tip designs with little or no gap in the material receiving area.

FIG. 32d is a side view of an exemplary positioner tip with little or no gap as the material receiving area.

FIG. 33 is a cross-sectional view of a wrapping and insertion device and positioning device loaded with material in the process of wrapping by use of a tapered opening and rotation of the positioner tip

FIG. 34 is a side view of an exemplary insertion system comprising a folded material, a positioning device, a wrapping/introducer tube, and a trocar cannula, where the introducer tube is inserted beyond the distal end of the trocar cannula.

FIG. 35 is a side view of an exemplary insertion system comprising a folded material, a positioning device, an introducer tube, and a trocar cannula, where the introducer tube is inserted in the trocar cannula, but proximal to the distal end of the trocar cannula.

FIG. 36 is a side view of an exemplary positioning device with a material orientation visual indicator 225.

FIG. 37 is a side view of an exemplary positioning device with adjustable joints.

FIG. 38 is a side view of an exemplary positioner (positioning device) with an adjustable length, e.g., the distal end telescopes into the shaft section and can lock into position so that the distal end of the positioner does not move backward relative to the shaft and handle of the positioner.

FIG. 39 is a perspective view of an embodiment of a material depth stop which can stabilize the projections by sliding axially along the projections.

FIG. 40 is a cross-sectional view of another embodiment of a positioner tip comprising curved members.

FIG. 41 is a cross-sectional view of yet another embodiment of a positioner tip having a material receiving area that is not centered along the axis of the distal tip.

### DETAILED DESCRIPTION

Approximately one million herniorrhaphy surgeries are conducted every year in the United States. These surgeries can be conducted either by open procedures or utilizing laparoscopic methods. Laparoscopic hernia repair procedures provide many benefits over open procedures. These benefits include decreased recovery times, lower infection rates, reduced post-procedure pain, and reduced incisional scarring Despite these significant advantages laparoscopic procedures, on average, require more procedural time to complete than open surgery.

During a laparoscopic procedure, three incisions are typically created in the abdomen, each being about 5 millimeters (mm) to about 15 mm in length. Through each incision a trocar cannula can be inserted and advanced into the abdomen through the abdominal wall. Once access to the abdominal cavity is gained, the inner trocar can be removed from the outer cannula, and the cannula can serve as an access port through which laparoscopic devices can be inserted. Cannulas also comprise an insufflation port/valve that can be connected to a gas source (e.g., carbon dioxide) to insufflate the abdominal cavity. During insufflation the abdominal cavity is inflated, which distends the muscular anterior abdominal wall from the viscera. The cavity formed provides a working space that enables for viewing and manipulation of laparoscopic devices therein.

Once insufflated, a defect can be located using anatomical markers and probing utilizing laparoscopic devices (e.g., grasper). Once located, closure of the defect can be achieved by reinforcing the abdominal wall with a repair material (e g., a hernia mesh). The repair material can be forced through the cannula. The repair material is then unfolded and positioned with grasper(s). Once in place on the anterior abdominal wall, the material can be secured (e.g., fixated) in place with a laparoscopic suturing or stapling device.

Although this procedure can be summarized in relatively few steps, the process of inserting and manipulating the repair material is time consuming, challenging and cumbersome for the physician. Manipulating the material extensively (which is required in the procedure without the benefit of the devices described herein) also presents the risk of injury to the patient by damage to tissues, vessels, and/or organs. Because procedural time represents the occupation of the many resources needed for a procedure (physician, assistants, other personnel, operating room occupancy, equipment, etc.) and often represents the most costly aspects of the operation, reduction of procedural time represent an opportunity to reduce procedural costs. The present disclosure will unveil a material delivery system that allows for faster and easier introduction, deployment, and positioning of material(s) (e.g., hernia prosthesis materials) and disclose methods for using the same.

Referring now to FIG. 1 that illustrates an oblique view of an exemplary positioning device generally designated 20. The positioning device comprises a shalt 26. Although the shaft 26 can comprise any cross-sectional geometry (e.g., rounded (such as circular, elliptical, and so forth), polygonal, irregular, as well as combinations comprising at least one of the foregoing), a geometry that is matable with various introducer device (e.g., cannulas, conduits, and so forth) and wrapping devices is desirable. The size of the shaft 26 is dependent upon the particular use for the positioning device. For example, the shaft 26 (which can be solid or hollow) can be about 4.0 millimeters (mm) to about 38.0 mm in outer diameter, have a wall thickness of greater than or equal to about 0.125 mm (e.g., about 0.125 mm to about 10.0 mm), and can have a length of greater than or equal to about 5.0 centimeters (cm).

Connected to shaft 26 can be a handle 28, which is configured to be held by a human hand. The handle 28 can comprise any geometry, such as a pistol-grip, syringe-type grip, tubular grind, and forth and can be connected to shaft 26 by any method (e.g., injection molding, welding, bonding, crimping., and so forth). For example, handle 28 can be injection molded from acrylonitrile-butadiene-styrene polymer and adhesively bonded to a stainless steel shaft 26. The shaft and handle can also be integral components fabricated of the same material.

Connected to the shaft 26 can be an optional secondary shaft 46, which has a diameter that is less than or equal to the shaft 26 diameter. If the secondary shaft 46 is smaller in diameter than the shaft 26, a tapered section 70 (i.e., a section that becomes gradually narrower) can connect the two components to inhibit the positioning device from snagging on any device into which it is inserted or on an organ or other body tissues after insertion into the body.

Connected to the shaft 26 or the secondary shaft 46 can be a flexible section 30, which is capable of deflecting. The flexible section 30 can comprise any flexible element (e.g., rod, tube, cable, coil, cylinder, and so forth), or a flexible member that is cut to allow for flexibility. Possible materials include polymers, metals, as well as combinations comprising at least one of the foregoing materials, e.g., a single-filar, pitched coil, having a circular cross-sectional geometry, a filar metallic component in conjunction with a flexible polymer, a flexible multi-filar cable, a flexible polymer solid rod or tubular section, and so forth. In addition, designs can be employed that comprise a non-round cross-sectional geometry to provide limited flexibility on one or more directions and increased flexibility in other directions. For example, a nickel-titanium alloy rod comprising an elliptical cross-sectional geometry can be utilized, wherein the elliptical cross section offers flexibility in a direction transverse the major axis and limited flexibility in a direction with the minor axis. Furthermore, the flexible section 30 can comprise a flexible polymer sheath (not shown) that can hinder bodily fluids from penetrating the flexible section 30, so as to allow for easier resterilization, and/or can prevent tissues from being pinched as the coil for example is manipulated within the body.

The connections joining the shaft 26, secondary shaft 46, and the flexible section 30 should be durable so that the sections do not decouple during use. Any method for connecting these sections can be employed, such as injection molding, adhesive, welding, and crimping, as well as combinations comprising at least one of the foregoing. Where desired, the connections of these sections can employ designs to maintain radial orientation of the sections to prevent independent rotation between sections. Examples of this design include integral transitions, keyed or mating flat surfaces between components, and so forth. Also where desired, designs can be employed that allow for the functionality of independent rotation; e.g., a device can have selectable independent rotation of components in one setting and be “locked” by selective actuation to prevent independent rotation in another setting.

Connected to the flexible section 30 can be a distal section 32, which is illustrated as a tubular design. The distal section 32 can comprise projection(s) of various cross-sectional configurations and a material receiving area to receive and releasably retain a material (e.g., a repair material), such as slot 34 that extends from the end of the tip section 32 along its length parallel with the tip section's axis. The dimensions of slot 34 can be dependent upon the material to be received in the material receiving area. For example, length of the slot 34 can be the entire length of the tip section 32, or any portion thereof, while the width can be equal to or less than the internal diameter of the tip section 32. Furthermore, the edges of the slot 34 can comprise a radius. It is envisioned that the distal end can have an interrupted material receiving area where there is a section adjacent to the distal receiving area that does not retain the material, and adjacent to that section is another material receiving area. The interruptions can comprise a section to allow flexibility of the distal end. The distal tip in any design can be flexible enough to be deformed by pressure exerted by the hand on the handle of the positioning device.

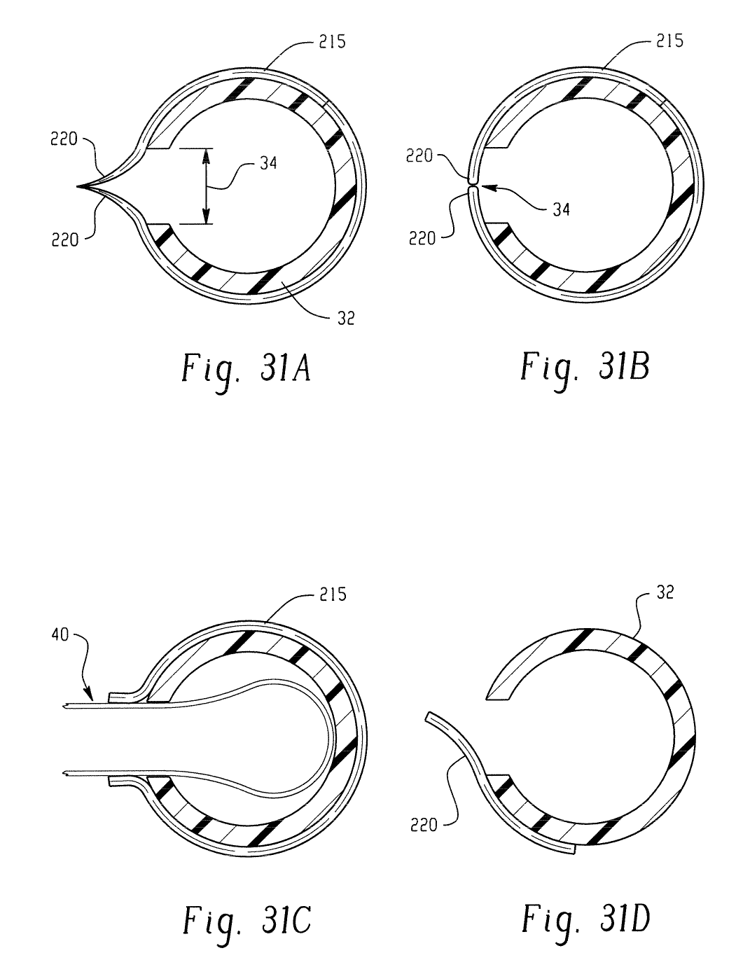

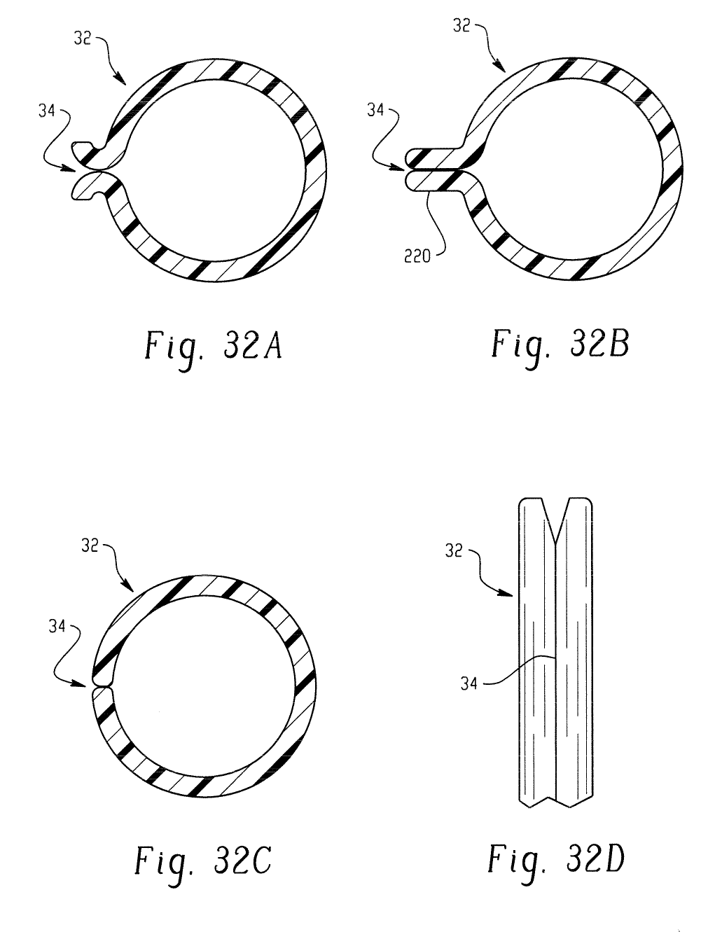

The material receiving area can have a distal opening that is larger at the very end and is reduced in dimension axially to facilitate facile insertion of material into the receiving area. Although the material receiving area is illustrated as a slot with an axis parallel to that of the tip section 32, it can comprise various geometries, such as a helical configuration having a clockwise or counter-clockwise rotation, an irregular configuration, and so forth. In addition, referring to FIGS. 31a-d, the material receiving area 34 can also be bounded by flexible element(s) 220 with no gap or with a gap equal to the outer dimension of the positioner tip for material insertion. The purpose of the flexible elements can assist in the releasable holding of the material (e.g., by means of friction) and/or can provide functionality in support of the folded and loaded material to aid in the positioning of the material in the unwrapped configuration once inside the body. The flexible elements are flexible enough to be deflected when the material is inserted into the material receiving area by hand without damaging the material.

The tip section 32 can be fabricated from materials such as those described in relation to the flexible section 30. For example, the tip section 32 can be fabricated from a stainless steel material with a slot 34 machined therein. The tip section can be joined to the flexible section 30 using a welding or other joining process.

In an optional configuration the positioning device can comprise a mechanism to allow powered movement (e.g., powered rotation) of the distal section 32 independently of the handle 28. As rotation of the positioning device can be used in various methods of operation, the ability to rotate the end in one or both directions at a set or variable speed by an active means (e.g., stored electrical energy in a battery, current from a utility outlet, and so forth), could provide added functionality for the device. Other options for rotation include mechanical rotation other than manually turning of the positioning device 20 on its axis; e.g., a spring loaded lever and a gear drive.

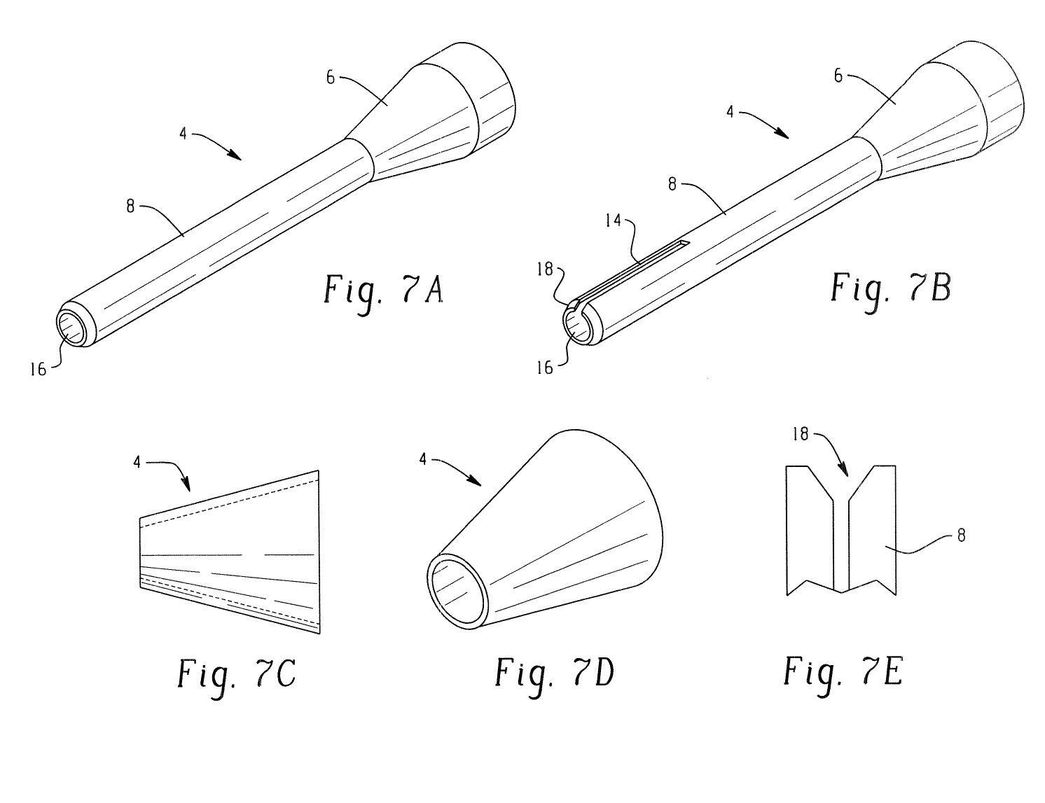

Referring now to FIG. 7a, an isometric view of an exemplary wrapping and introducer tube device, generally designated 4, is illustrated. Introducer tube 4 comprises a tube 8, which comprises a lumen 16 that extends the length of the introducer tube 4. The cross-sectional geometry of the tube 8 can be of any geometry (e.g., rounded (e.g., circular, elliptical, and so forth), polygonal, irregular, as well as combinations comprising at least one of the foregoing). In some embodiments, tube 8 can be about 4.0 mm to about 40.0 mm in outer diameter (OD), comprise a wall thickness of about 0.2 mm to about 6.0 mm, and can be equal to or greater than about 1 centimeter (cm) in length.

Disposed on the proximal end of the tube 8 can be an element 6. The element 6 can comprise an internal geometry that comprises an internal diameter that is equal to or greater than the internal diameter of the lumen 16, and coaxial and contiguous therewith. The internal geometry preferably comprises a taper with a larger diameter opening at the proximal end of the element 6, and a reduction in diameter distally toward the inside of the device, for aiding the insertion of devices into the introducer tube 4, the positioning the device loaded with surgical material, and/or facilitating the wrapping of material that has been loaded in to the positioning device when aligned and loaded into that end of the introducer. To provide a seal around devices that are inserted into the introducer tube 4, the element 6 can comprise a valve (not shown) that is capable of maintaining insufflation pressures of less than or equal to about 20 millimeters per mercury (mm/Hg), or more specifically less than or equal to 40 mm/hg, or even more specifically, less than or equal to 60 mm/hg. The valve can be constructed of an elastic polymer such as silicone, polyurethane, and so forth, as well as combinations comprising at least one of the foregoing, and can comprise any geometry (e.g., duck-bill, annular, flap, and so forth). The introducer tube can also comprise a port valve capable of being connected to a gas supply for insufflation, therefore enabling the introducer to function (and be used) as a trocar cannula. Where the introducer tube can be used as a trocar cannula, a slot can be employed for an optional method of wrapping the material loaded into the positioning device.

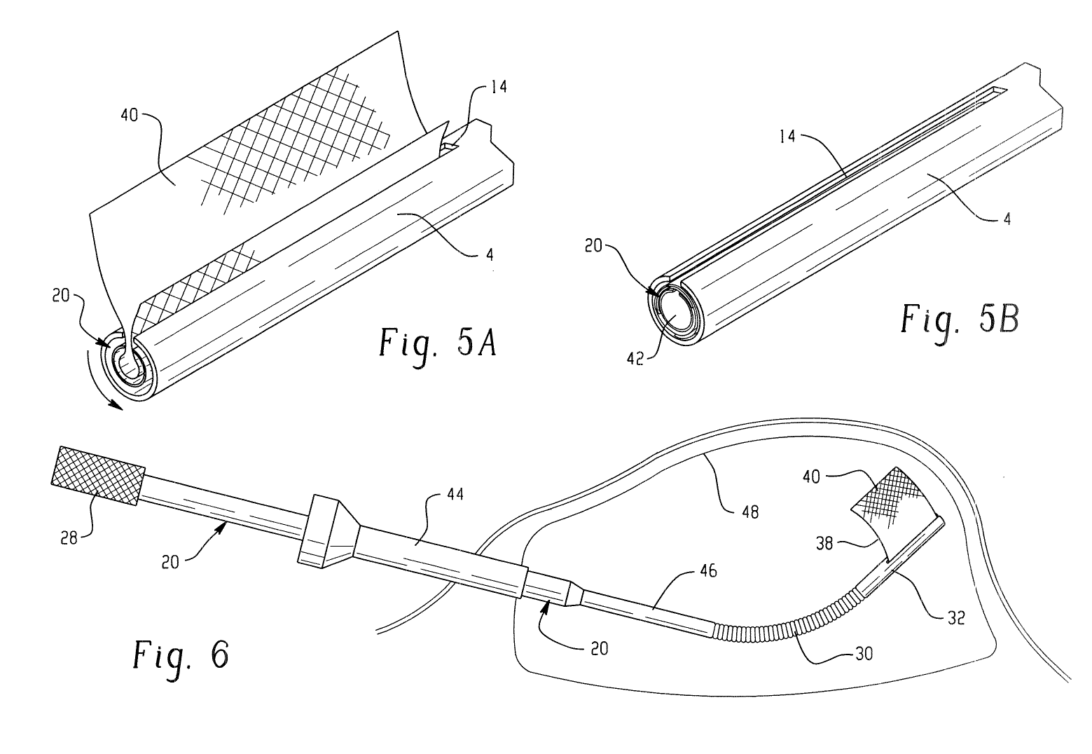

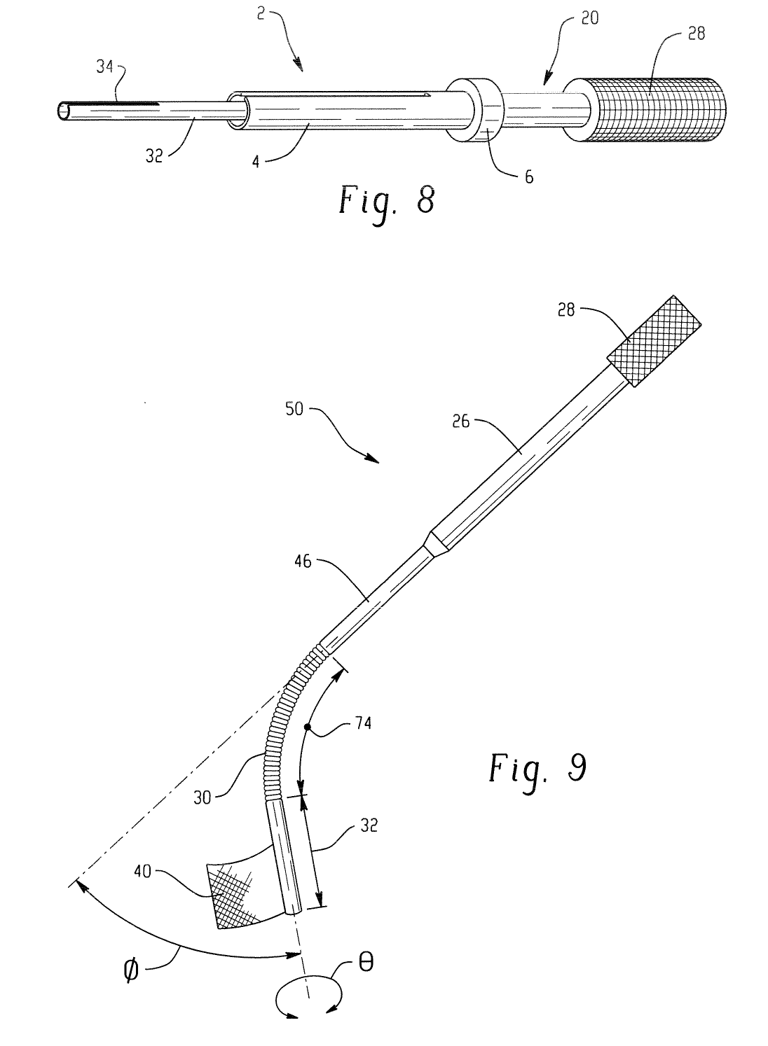

Referring to FIG. 7b, an isometric view of an exemplary wrapping/introducer device (e.g., tube) is provided. On the distal end of the tube 8 a material receiving area (e.g., a slot) 14 can extend from the end of the tube 8 to any length along the tube 8. The material receiving area can comprise a width that is less than or equal to the inside diameter of the lumen 16 and can comprise a radius and/or chamfered edges. Also disposed on the distal end of the tube 8 can be an optional taper 18. This material receiving area allows insertion of the folded material loaded in the positioning device (which has previously been inserted in the introducer tube), to be inserted axially when the loaded material in the positioning device is in alignment with the material receiving area. Referring now to FIG. 8, an oblique view of an exemplary material delivery system, generally designated 2, is illustrated. The material delivery system 2 comprises an introducer tube 4 through which a positioning device 20 has been inserted. In this configuration, the positioning device 20 can rotate freely within the introducer tube 4. When the positioning device is rotated relative to the introducer tube, the material is drawn inside the inner diameter of the introducer tube and forms a rolled configuration around the distal end of the positioning device (See FIGS. 5a and b). The positioning device 20 can also translate therein, restricted only by interference between handle 28 and element 6, which limits the travel of the positioning device 20 into the introducer tube 4.

Referring to FIGS. 7c and 7d, the wrapping/introducer as well as combinations comprising at least one of the foregoing device can comprise little or no tubular portion. A generally tapered or inverse conical shape is used for wrapping and insertion of the wrapped material into a trocar cannula or directly in to the body.

The introducer tube 4 can be constructed utilizing polymers (e.g., polytetrafluoroethylene, polyethylene, acrylonitrile-butadiene-styrene and so forth), metals (e.g., aluminum, titanium, stainless steel, and so forth), metallic alloys (nickel-titanium and so forth), and so forth. The exact materials chosen for each element will depend on properties desired and manufacturing methods employed (e.g., rigidity, lubriciousness, manufacturing method). For example, in one embodiment, tube 8 can be extruded from polytetrafluoroethylene that is cut to a desired length. Slot 14 can be stamped into the distal end of the tube 8 and a grinding operation can be employed to radius the slot's edges as well as form a taper 18. An acrylonitrile-butadiene-styrene element 6 can be insert injection molded over the proximal end of the tube 8 (not shown), wherein the proximal end of the tube 8 can be flared to provide additional retention between the tube 8 and the element 6. Any number of materials, fabrication and assembly features and means can be used to produce this instrument.

As described briefly above, during some defect repair procedures that do not employ the device described herein, the material is folded and forcibly introduced through a cannula without any means to control, orient, hold, or deliver the material once inserted into the body. This delivery method poses the potential of the material to become entrapped within the cannula and/or damaged during the process. In addition, once the material is introduced, it exits the distal end of the cannula and is unfolded and carried to the defect site using graspers and/or an alternative laparoscopic tool. Once the material has been transported to the repair site, graspers can be used to position the material so a staple, suture, or the like, can be placed therein to secure the material. The additional instruments like metallic jaw graspers which are needed to grasp, manipulate unfold, hold, and orient the material, can cause injury to tissues, vessels and organs in the process of repeatedly releasing and grasping the material as required to unfold and orient the material inside the body. Not only can the material incur damage during this procedure, but it also poses a challenging and time consuming procedure for the physician.

The present device system facilitates the introduction, deployment, positioning and application of surgical repair material, such as a hernia mesh. The operation of the device disclosed herein alleviates the potential of damage to the material during introduction, risk of injury, can provide for easier transport and positioning of the material to and at the defect site, can preserve a specific orientation of the material in relation to the application site (eliminating the need for graspers), can be used to directly apply the material to the defect site, and can significantly reduce procedural time.

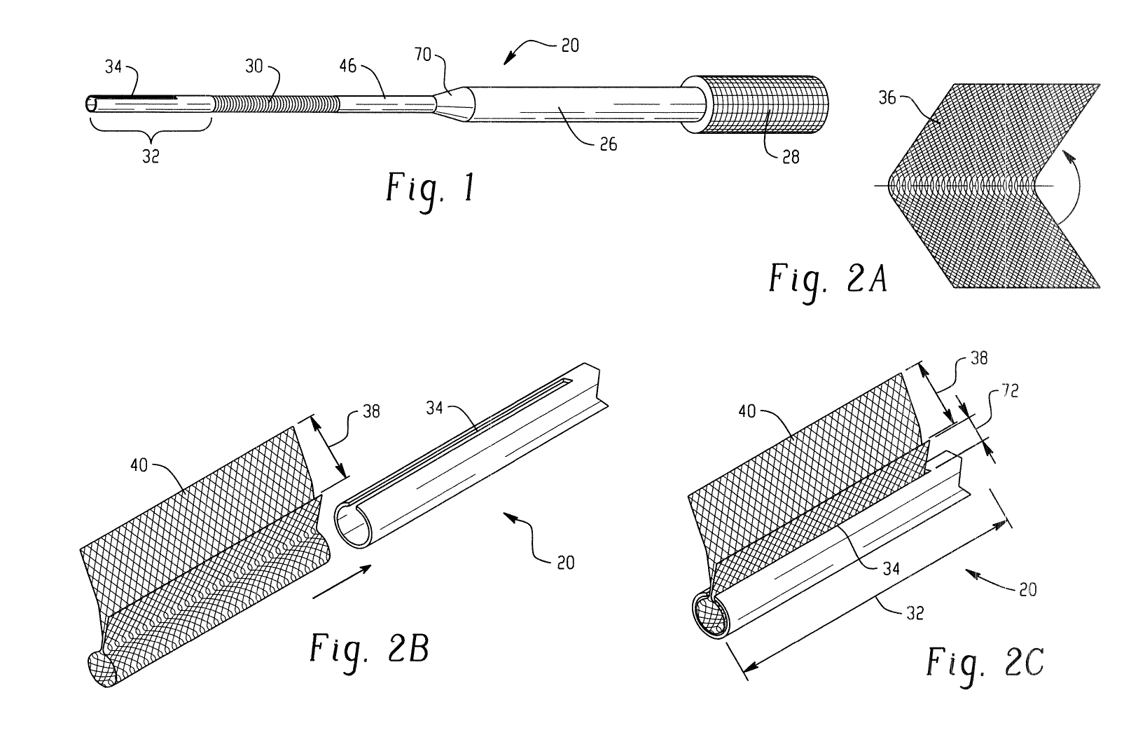

The process of using the material delivery system (e.g., positioning device) preferably begins with bending or laying upon itself to create a curve (e.g., folding) a material, as illustrated in FIG. 2a. In the illustration, a repair material 36 is folded over onto itself to form a folded repair material 40 (See FIG. 2b). The fold of folded repair material (e.g., mesh) 40 can then be inserted into the material receiving area 34. The folded repair material 40 is then advanced until it contacts the end of the slot 34, as illustrated in FIG. 2c. If the slot 34 is longer than the folded repair material 40, the distal end of the material can be aligned with the distal end of the slot 34. The purpose of folding the material 36 is that the folded portion within the tip section 34 acts to releasably secure the folded repair material 40 in the slot 34. In addition, folding the material 36 off-center, less than in half, such as folding the material 36 at about one-third or even at about one-quarter or less of the material's length can provide a single layer lap 38 that can be easily fixated (stapled, sutured, or the like) to a defect site. It should be noted that is undesirable to fixate the material 36 in a position other than the single layer flap 38 when the material 36 is folded for the reason the folded repair material 40 could not be unfolded thereafter. Therefore, the material can be folded in any position along the material's length that will provide a flap 38 and an excess portion 72 that extend out of the slot 34. The folding of the material also allows the multiple layers of material to provide additional body or support where needed to allow the material to generally extend more radially outward from the axis of the positioning device which enhances the holding of the material against the area to which it will be affixed. Material folded in this manner also simplifies spiral wrapping and unwrapping. Also, although the folded repair material 40 is illustrated as folded parallel to an edge of the repair material 36, it is envisioned the repair material 36 can be folded in any orientation. It is also envisioned that the material can be additionally folded, more than once in order to achieve the flap portion that would allow releasable holding, support, positioning and the ability to fix the material to the body and release from the positioning device.

The slot 34 is intended to be configurable to allow for the use of any mesh, 36 material, size, or geometry. In one embodiment, the width and length of the slot 34 can be specifically configured to releasably secure a specific material 36. In another embodiment, the material slot 36 can be configured to comprise a standard length and width that is capable of accepting a range of material 36 sizes.

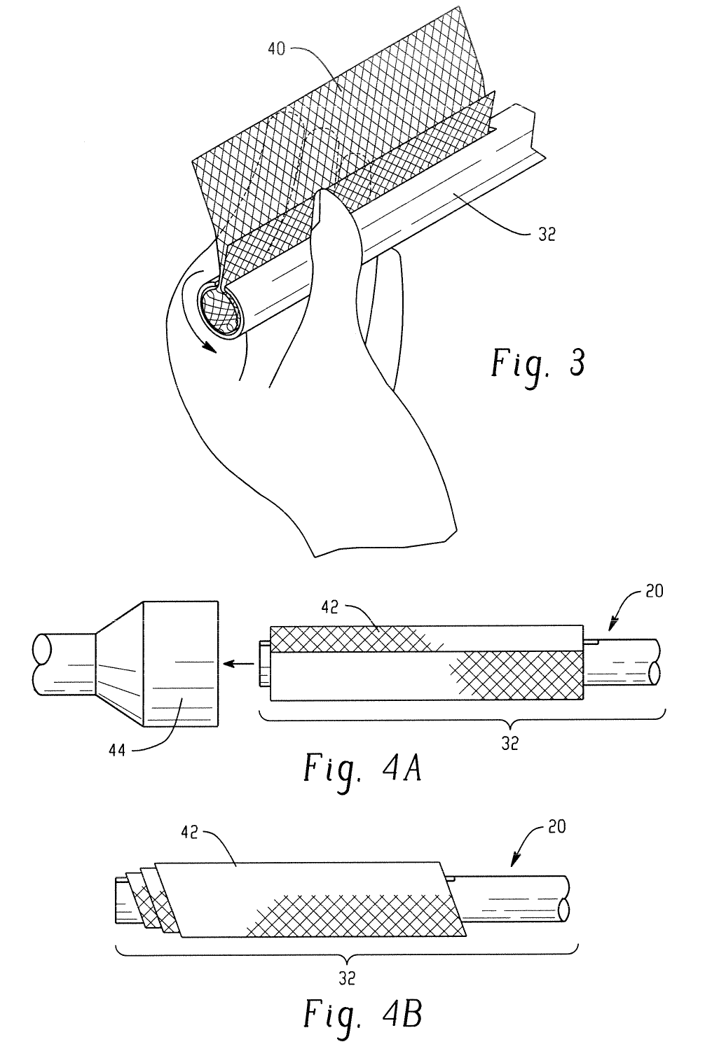

In one method of using the material delivery system, the loaded, folded repair material 40 can then be rotated using one hand while wrapping the folded repair material 40 around the tip section 32 of the positioning device with another hand, as partially illustrated in FIG. 3. During a surgical procedure, the operator of the device will have sterile gloves. Hence, it is with a gloved hand that the wrapping of the material can be accomplished. The fingers and/or palm can be used to guide the material in to a spiral configuration as the distal tip is rotated. It is envisioned that an optional barrier to aid in forming of the material can be placed between the hand and the material to aid in shaping, reduce friction, and/or to prevent contact between the material being rolled and the gloved hand. Once the folded repair material 40 is wrapped around the tip section 32, the wrapped repair material 4? can be inserted into the cannula (e.g., trocar cannula, introducer devices and so forth) 44 to gain access into the abdominal cavity, as depicted in FIG. 4a. FIG. 4b depicts a pitched, spiral configuration that can be advantageous in the insertion of in to the opening, internal features and inner diameter of medical cannulas like those described herein. The pitched configuration presents the distal end of the material in a lower profile that tapers to a higher profile as the material is wound proximally. This profile, like any tapered geometry, can more easily be introduced where close fitting components are encountered. The pitched configuration can be created by causing an angulation of the material as the material is wrapped, and by loading the material at an angle that is not exactly normal to the axis of the positioning device.

At this point, after insertion into the body through a cannula, the wrapped repair material 42 can begin to unwrap itself. If the material does not unwrap, or at least unwrap enough to ensure the flap 38 is accessible for fixation, the wrapped repair material 42 can be unwrapped by rotating the positioning device 20. Once the folded repair material 40 has been unwrapped, the positioning device 20 can be advanced and guided to the treatment site under the direction of laparoscopic monitoring per physician preference while accounting for the variables associated with the laparoscopic surgery (e.g., approach angles, distances, obstructions, and so forth). Once the folded repair material 40 has been positioned close to the defect site, the positioning device 20, and trocar cannula, can be manipulated to influence the flexible section 30 to bend (if desired) to attain a desired placement of the folded repair material 40, as illustrated in FIG. 6. A “desired placement” can be interpreted as placement of the folded repair material 40 wherein the flap 38 is positioned in a location relative to the defect and abdominal wall 48 wherein the flap 38 can be fixated to the abdominal wall 48. Once positioned, a laparoscopic fixating device (e.g., stapler) can be inserted through a separate cannula 44 and utilized to secure the flap 38 of the folded repair material 40 to the abdominal wall. Once fixated, the positioning device can be retracted to cause the material to deploy from the slot 34, and further fixated as desired by the physician. Once the folded repair material 40 has been deployed the positioning device 20, a fixation device, and/or a combination of devices can be utilized to manipulate the material 36 to a desired position, where it can be fixated. Material deployment can be aided by locating the end of the trocar cannula in the vicinity of the intended deployment. After it has been positioned, the positioning device can be moved axially toward the end of the trocar cannula thereby stripping the unfurled material from the releasable hold of the positioning device. In other methods where other cannulas are used, those cannulas can be used to manipulate material and also to strip the material from the positioning device in the same way. Removal in the absence of fixing to tissue can also be accomplished by using a grasping device to remove from the positioning device. Material deployment without prior fixation can also be completed using the methods described above.

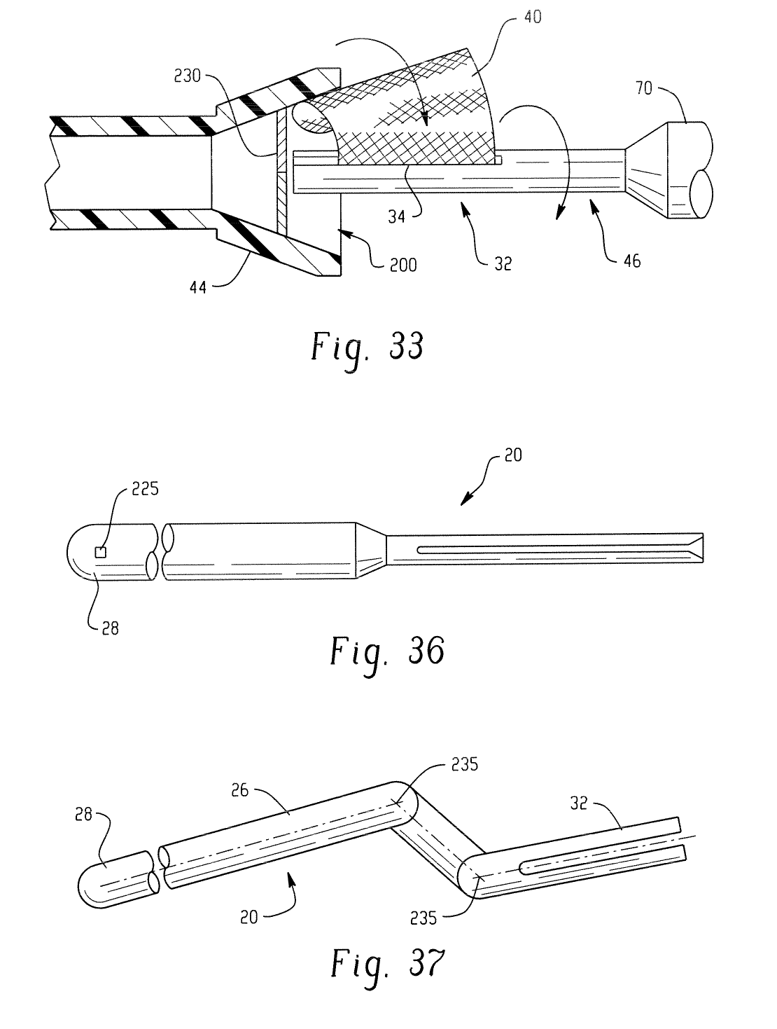

Another method of wrapping material loaded in the positioning device for insertion in to a body uses a trocar cannula with a tapered opening 200 as is illustrated in FIG. 33. The tapered opening is of sufficient geometry to shape the material into a generally spiral configuration as it is advanced in to the end of the trocar cannula. This is accomplished when the material loaded in the positioning device is advanced to the opening of the wrapping trocar. The leading edge of the material is oriented in a position whereby it can enter the proximal trocar opening in conjunction with rotation of the end of the positioning device. This allows the wrapping of the material 40 around the tip section 32. Once the folded repair material 40 is wrapped around the tip section 32, the wrapped repair material can be inserted into the cannula 44 to gain access into the abdominal cavity. (See FIG. 6) It is envisioned the positioning device can continue to be rotated in the direction that it was spirally wrapped around the end of the positioning device to encourage the wrapped repair material to advance through the cannula's valve 230, if present. Once introduced through the valve 230, the wrapped repair material can be advanced through the cannula 44 (while optionally rotating), out of the distal end of the trocar cannula 44 and into the abdominal cavity.

Following are two methods of using the material delivery system for introducing material using a wrapping/introducer device to insert the material in through an a trocar cannula that has been previously placed in the body. A material 36 can be folded as illustrated in FIG. 2a, and loaded into the material receiving area 34 of the positioning device as illustrated in FIGS. 2b and 2c. The folder repair material 40 can then be wrapped by inserting it into an end of a wrapping/introducer tube device as shown it FIGS. 7a and 33, with an opening configured to receive unwrapped material and form the material to a spirally wrapped configuration, e.g., using the tapered opening 200 which is larger at the end of insertion, and narrows as the material is advanced inside. The material loaded in to the positioning device can be inserted distally and rotated simultaneously thereby allowing the device to wrap the material around the distal end of the positioning device. The positioning device has an internal geometry that terminates in an inner diameter of a predetermined dimension that allows insertion of the loaded, wrapped material into the body.

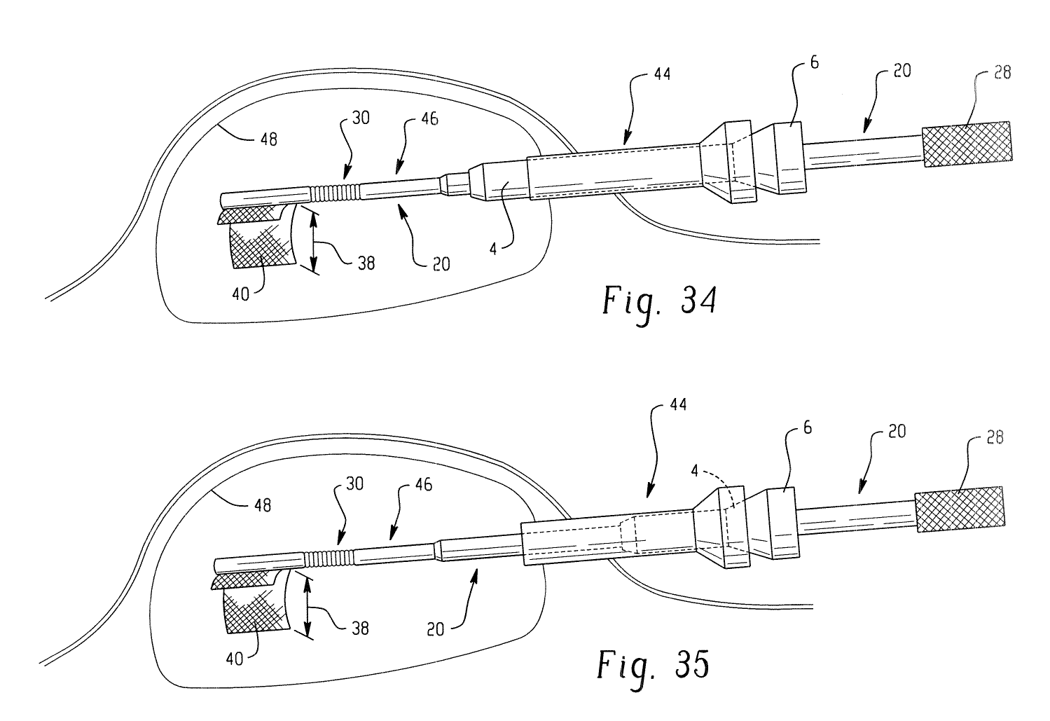

In one embodiment, the method of using a wrapping/introducer device to insert the material in through an in-place trocar cannula comprises inserting the wrapped material inside device 4 into cannula 44 that has been previously placed in the body to gain access into the abdominal cavity (see FIG. 34). The wrapping/introducer device 4, with the positioning device and wrapped material within, is advanced through the cannula 44, and out the distal end or to near the distal end. The positioning device is then advanced beyond the distal end of both the cannula and the wrapping/introducer cannula and into the abdominal cavity. The material can then be unwound, positioned, and fixated. Optionally, the introducer tube 4 can also be manipulated as needed to assist in positioning the folded material.

In another embodiment, the method of using the wrapping/introducer device to insert the material in through an in-place trocar cannula comprises inserting into a trocar cannula, a wrapped repair material that is inside the wrapping/introducer device 4, wherein the trocar cannula that has been previously placed into the body to gain access into the abdominal cavity (see FIG. 35). The wrapping/introducer device 4 can have the geometry to allow insertion in the trocar cannula, but not completely through the length of the trocar cannula. The positioning device can be rotated to encourage the wrapped repair material to advance through the cannula and into the abdominal cavity where it can be unwound, position, and fixated.

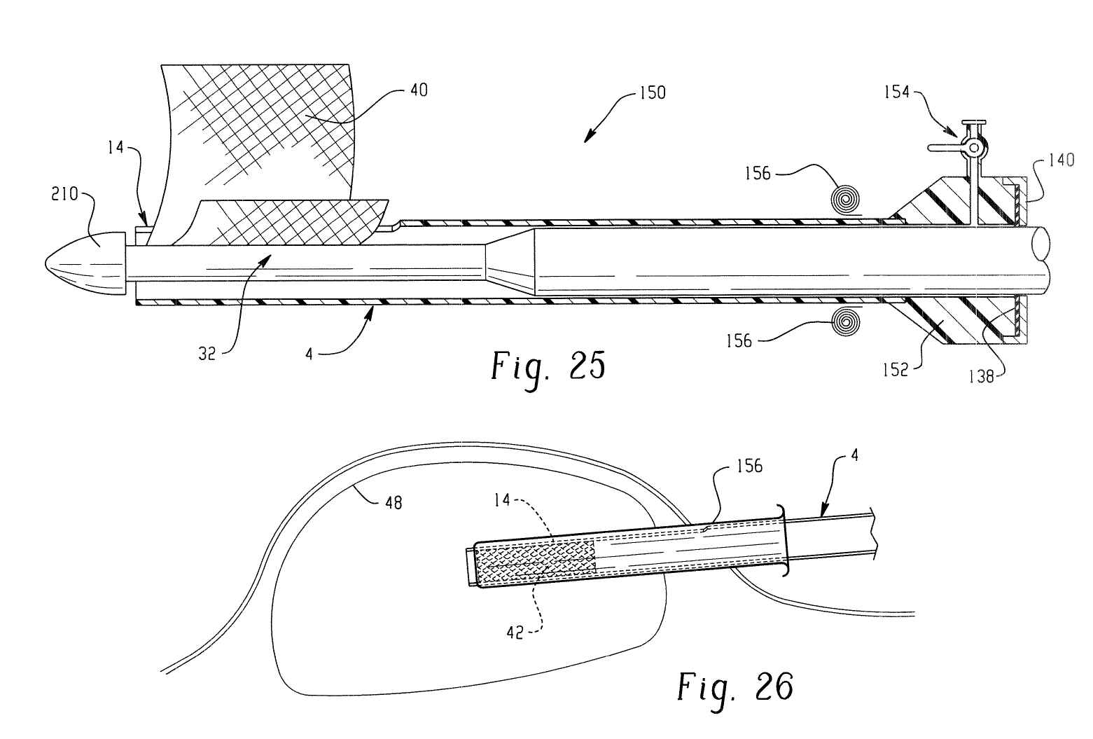

Optionally, the above methods can be performed with the wrapping/introducer device can be disposed in the trocar cannula before the cannula is positioned in the body. The distal end of the positioning device can be shaped especially for insertion directly into an incision in the body (e.g., conically) and projects beyond the distal end of the trocar cannula upon insertion in order to facilitate insertion of the loaded positioning device and trocar assembly in to the body. An exemplary positioner tip of this kind is shown in FIGS. 30a and 30b, and an exemplary material insertion system using such a positioning device is shown in FIG. 25.

In the other method, a specialized trocar cannula is provided that has a slot 14 in the distal end (see FIG. 24) that facilitates wrapping of material that has been loaded in the positioning device. After the folded repair material 40 has been loaded into the slot 34, the slots 34 and 14 can be aligned by rotating the positioning device 20 within the introducer tube 4. Once aligned, the flap 38 and excess portion 72 can be retracted into the slot 14 by retracting the positioning device 20 into the introducer tube 4 until the distal end of the folded repair material 40 is aligned with the distal end of the slot 14, as illustrated in FIG. 5a. Once the folded repair material 40 has been retracted into the slot, the positioning device 20 can be rotated (clockwise or counter-clockwise) 4, as illustrated by the directional arrow in FIG. 5a, to wind the folded repair material 40 around the tip section 32 of the positioning device 20 to form a wrapped repair material 42, as depicted in FIG. 5b. Once the wrapped repair material 42 is within the specialized trocar cannula 4, the material delivery system can be introduced into the body through an incision. Once inserted, the loaded positioning device can be advanced until the wrapped material held by the positioning device extends past the distal end of the cannula 44. This can be monitored by laparoscopic visualization. The material can be unwrapped, delivered, positioned and fixed as described previously.

Another method of use is to load the material into the positioning device by wrapping the material manually as shown in an exemplary FIG. 3, or by using a wrapping device and directly inserting the positioning device and material into the body through an incision. In an optional positioning device design for this method, the distal end of the positioning device can be shaped especially for insertion directly into an incision in the body. (See FIGS. 30a and 30b)

Another method of use involves loading material into the positioning device, wrapping the material and inserting the wrapped material through a device located over an opening through an abdominal wall.

An additional method is to insert the material loaded in the positioning device, not wrapped, in to a device other than a trocar cannula that has a tapered opening of geometry that facilitates complete wrapping or completion of initiated wrapping of the material when the positioning device is rotated relative to the device and advanced.

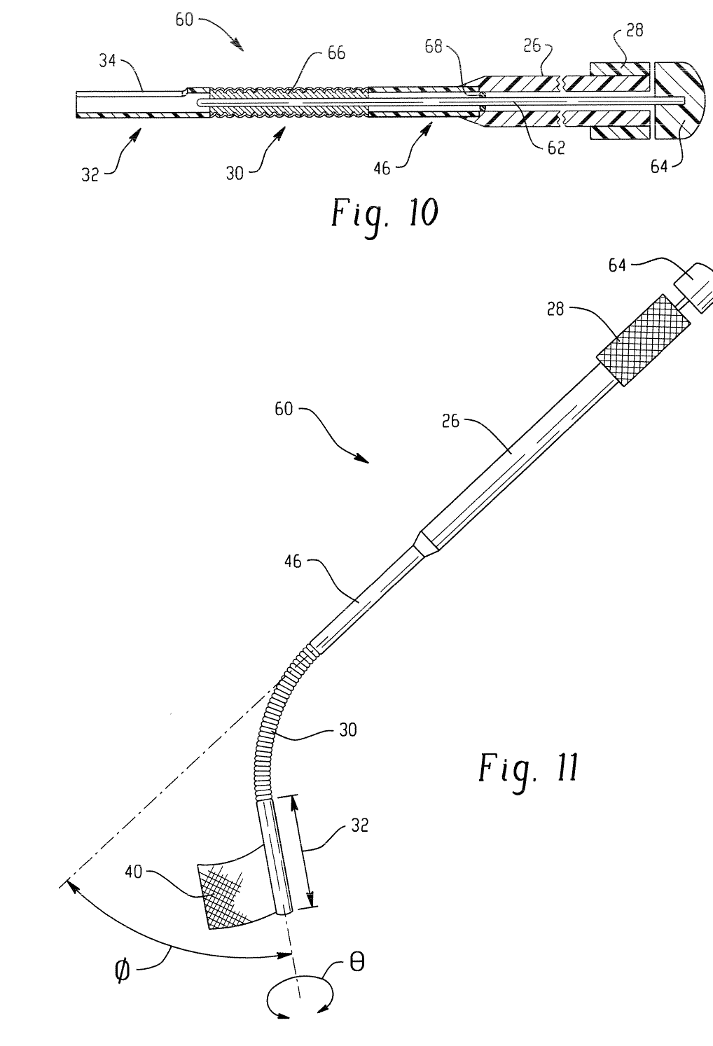

Referring now to FIG. 9, an isometric view of an exemplary curved positioning device, generally designated 50, is illustrated. In the illustration, the curved positioning device 50 is shown comprising a curve in the flexible section 30 of the device, which comprises an angle φ, which can be any angle that is less than or equal to 180°. The curve angle φ can enable a user to position the folded repair material 40 in a desirable position with greater ease. Curved positioning devices 50 can be manufactured with the specific curve as desired by the physician. For example, a first device can comprise a curve angle of about 30°, a second device can comprise a curve angle of about 45°, and a third device can comprise a curve angle of about 60°. In addition, although not shown, the curve can be complex and comprise multiple curves in a similar plane, or on various planes with respect to one another. Furthermore, it is envisioned the tip section 32 can be configured in a multitude of rotated configurations with respect to the curved section so that the direction at which the folded repair material 40 exits the slot 34 can be configurable to physician preference. For example, in FIG. 9, the rotation angle θ can be configured to a first desirable angle θ, however a second device can comprise a different angle θ.

Referring now to FIG. 10, a cross-sectional side view of an exemplary rotatable tip positioning device, generally designated 60, is illustrated. In the illustration, the rotatable tip positioning device 60 can comprise similar elements to the positioning device 20. More specifically, the rotatable tip positioning device 60 comprises a handle 28 that can be connected to a shaft 26, which can be connected to an optional secondary shaft 46. The secondary shaft can be attached to a flexible section 30. Disposed within the shaft 26, secondary shaft 46, handle 28, and flexible section 30 can be an internal lumen in which a wire (e.g., a cable, filament, line, and so forth) 62 can be disposed that is free to rotate therein. Connected to the wire 62 on its proximal end can be a knob (e.g., a rounded handle, lever, and so forth) 64, wherein the knob 64 can control the rotation of wire 62. Connected to the wire 62 on it distal end is a tip section 32, which can rotate as a result of rotation of knob 64 via wire 62 with respect to handle 28, shaft 26 and flexible section 30. The tip section 32 can comprises a slot 34, which is capable of accepting a folded repair material 40.

The placement of the material in the positioning device independent of the axis of the handle can be achieved with adjustable joint(s) 235 as show in FIG. 37. These joints 235 can be adjustable through controls (e.g., manual controls) such as anchored wires located just distal to the joint that exits the handle.

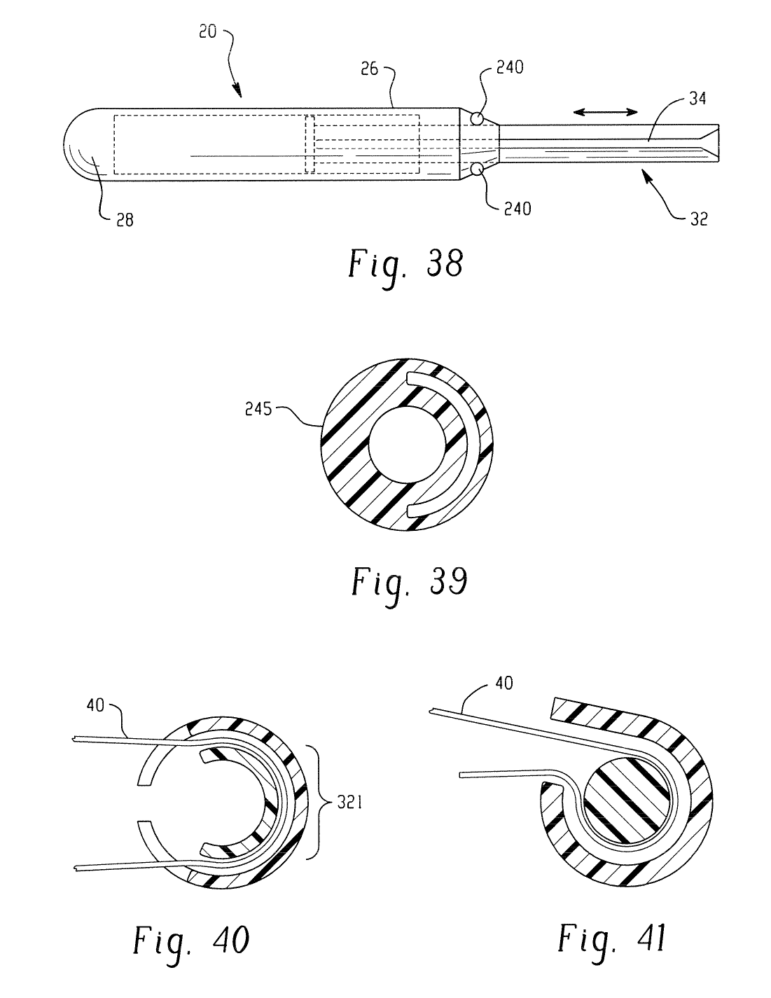

Various other embodiments of the positioning device are illustrated in FIGS. 38-41. FIG. 38 is a side view of an exemplary positioner (positioning device) with an adjustable length, e.g., the distal end telescopes into the shaft section and can lock into position so that the distal end of the positioner does not move backward relative to the shaft and handle of the positioner. FIG. 39 is a perspective view of an embodiment of a material depth stop which can stabilize the projections by sliding axially along the projections. FIG. 40 is a cross-sectional view of another embodiment of a positioner tip comprising curved members. FIG. 41 is a cross-sectional view of yet another embodiment of a positioner tip having a material receiving area that is not centered along the axis of the distal tip.

The junction between the flexible section 30 and the tip section 34 can employ washers, bushings, bearings, grommets, and so forth, to provide minimal resistance to rotation of the tip section 32, and provide a seal that is capable of preventing fluids (e.g., carbon dioxide, blood, irrigation fluids, and so forth) from advancing through the junction and up the internal lumen within the flexible section 30. Furthermore, flexible section 30 can comprise an optional barrier layer 66 that is capable of preventing the previously discussed fluids from advancing into the devices internal lumen if a fluid permeable flexibly section is employed, such as coil or cable. An optional polymer o-ring 68 (e.g., urethane, silicone) can be integrated into the device to prevent fluids from advancing from the abdominal cavity out of the handle 28.

The knob 64 can comprise any design that is capable of rotating the wire 62. A design that is similar in size and geometry to the handle 28 can be employed for example. The knob 64 and/or handle 28 can comprise a locking mechanism that is capable of locking the rotation of the handle 64 and/or wire 62 once a desirable rotation angle has been achieved.

The wire 62 can comprise a single or multi-filar cable comprising polymeric materials (e.g., acetal, polyethylene, polyamide) and/or metals (stainless steel), metallic alloys (nickel titanium), and so forth. The cable can also be coated with lubricous coatings (e.g., fluorinated polymer coatings, or shrink-tubing) to allow for smooth rotation. In one embodiment a 0.025 inch six-filar cable can be employed with a polytetrafluoroethylene-hexafluoropropylene copolymer (FEP) shrink tube disposed thereon.

The rotatable tip positioning device 60 can be constructed using common methods and materials that facilitate ease of manufacture and durability. For example, the handle 28 can be insert injection molded from a polyetherimide on a shaft 26 machined from stainless steel. Further, a silicone o-ring 68 can be inserted into the shaft 26 and the flexible section 30, comprising a single filar stainless steel spring coil can be welded to the shaft 26. A torque-coil comprising a counter-clockwise/clock-wise/counter-clockwise configuration of four-filar coils can be employed as the wire 62, which can be welded onto a stainless steel tip section 32, a polyethylene shrink tube can then be shrunk onto the torque-coil and the assembly can be inserted into the distal end of the flexible section 30 and advanced through the handle. A polyetherimide knob 64 produced from an injection molding process can then be adhesively bonded to the portion of the wire 62 extending from the handle 28.

The rotatable tip positioning device 60 is capable of comprising a curved distal section, as illustrated in FIG. 11 by angle φ in use, once the folded repair material 40 is positioned at or about the defect, the operator can rotate the knob 64, which will result in a variation of the tip section's rotation angle θ, thereby providing easier placement of the folded repair material 40.

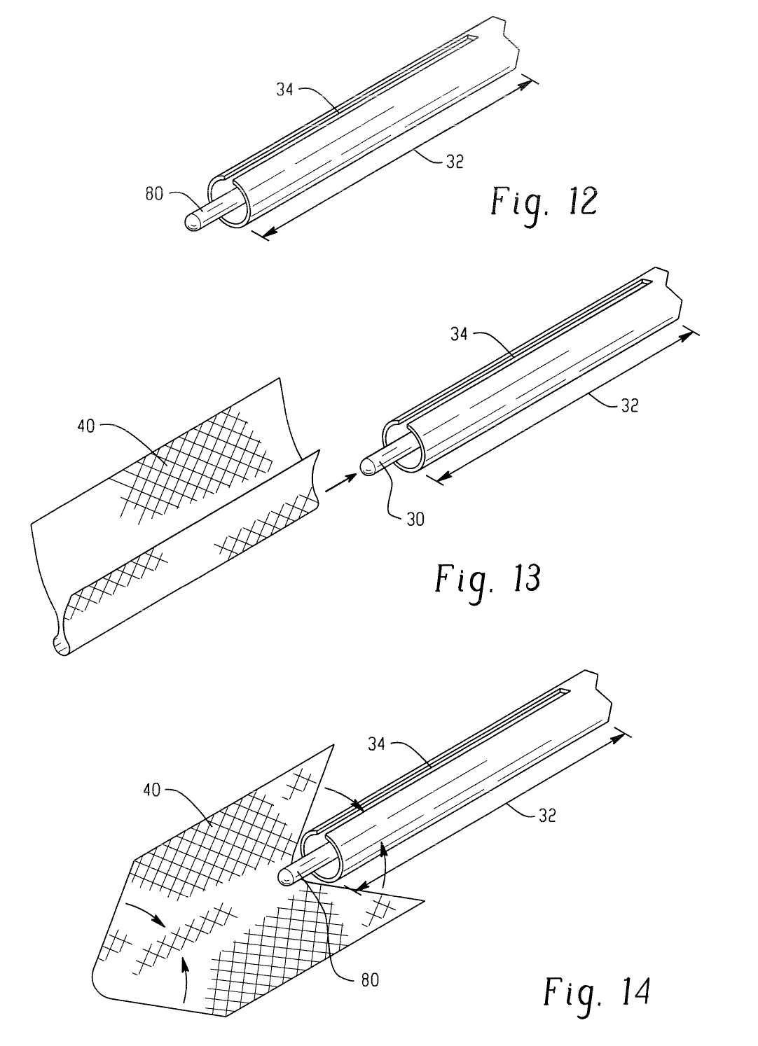

Referring now to FIG. 12, a partial isometric view of an exemplary tip section 32 comprising a loading pin 80 is illustrated. A loading pin 80 can be disposed within the internal diameter of a tip section 32 to assist in loading a folded repair material 40 within a slot 34, e.g., the pin can engage the material and facilitate loading thereof (see FIG. 16b). The loading pin 80 can comprise any shape (e.g., round, elliptical, polygonal, irregular, and so forth) and comprise any material (e.g., metal, polymer, metallic alloy). The loading pin 80 can any outer diameter that allows a folded repair material 40 to fit over the loading pin 80 and within the tip section 32. The length of the loading pin can extend to at or near the distal end of the tip section 32 or can extend there beyond. Furthermore, the loading pin 80 can be positioned in any configuration within the tip section 32, however a coaxial configuration can be produced as well. Possible loading pins include friction pin(s), retractable pin(s), atraumatic pin(s), rotatable pin(s), and combinations comprising at least one of the foregoing pins.

Loading pin 80 can be utilized as an aid while loading a folded repair material 40 into the slot 34. More specifically, a sheet of material 36 can be folded by hand and inserted onto the loading pin 80, as illustrated in FIG. 13, or the material 36 can be folded around loading pin 80, as illustrated in FIG. 14, and inserted into slot 34. The loading pin 80 can be fixated to the inside diameter to the tip section 32 in a location that does not interfere with the capability of loading the folded repair material 40 into the slot 34.

In another embodiment, the loading pin 80 can be a separate from the tip section 32 and utilized as a tool to load the folded repair material 40 into the slot 34 and then discarded at some point after loading. In this embodiment, a material can be folded around a loading pin 80 that is separated from the tip section 32, to form a folded repair material 40. The end of the loading pin 80 can then be inserted into the distal end of the tip section 32 and the flap 38 and the excess portion 72 of the folded repair material 40 can be inserted into the slot 34. The loading pin 80 can then be removed from the tip section 34 anytime after the loading of the material and discarded.

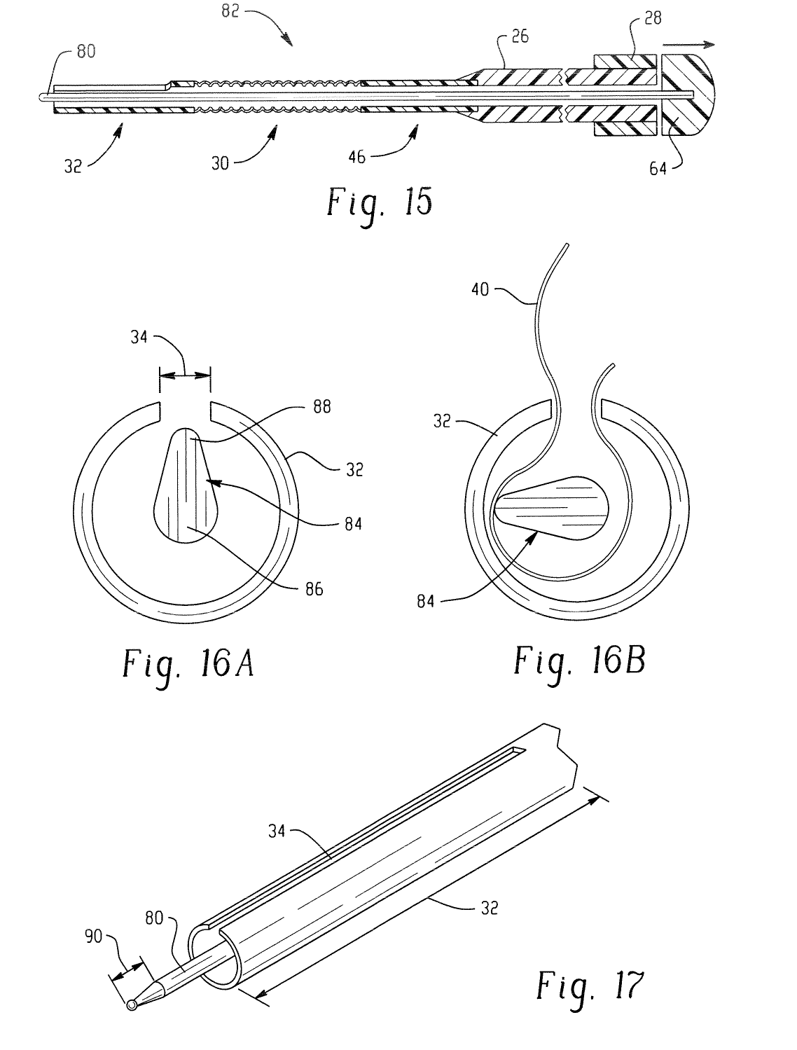

Referring now to FIG. 15, a cross-sectional side view of an exemplary retractable pin positioning device, generally designated 82, is illustrated. The retractable pin positioning device 82 comprises several elements similar to those employed on the rotatable tip positioning device 60. More specifically, the retractable pin positioning device 82 comprises a handle 28 that can be connected to a shaft 26. Shaft 26 can be connected to a secondary shaft 46. The secondary shaft 46 can be attached to a flexible section 30. Disposed within the shaft 26, secondary shaft 46, handle 28, and flexible section 30 can be an internal lumen in which a loading pin 80 can be disposed, which is free to translate therein. Connected to the loading pin 80 on the proximal section 12 is a knob 64, wherein knob 64 can control the translation of loading pin 80. The distal end of the loading pin 80 extends coaxially through the tip section 32 and extends past the distal end of the tip section 32. The tip section 32 can comprises a slot 34, which is capable of accepting a folded repair material 40 that is assembled onto the loading pin 80.

During use of the retractable pin positioning device 82, a material 36 can be loaded onto the loading pin 80 via the procedure discussed with respect to FIGS. 13 and 14. After loading of the folded repair material 40 into the slot 34, the folded repair material 40 can be retracted into a slot 14 via the procedures associated with FIG. 5a, and formed into a wrapped repair material 42 employing the procedures associated with FIGS. 5a and 5b. The wrapped material 52 can then be inserted into the abdominal cavity and positioned on or near the defect utilizing any method described above. Prior to deploying the folded repair material 40 however, the loading pin 80 is retracted by pulling knob 64 to a position that enables the folded repair material 40 the ability to deploy without interference from the loading pin 80. This position can be indicated by markings on the length of the loading pin 80 that are observable by the operator, or by completely removing the loading pin 80 from the positioning device 20.

The retractable pin positioning device 82 can be constructed using common methods and materials that can facilitate ease of manufacture and durability. In one embodiment it is envisioned the handle 28 is insert injection molded onto a polymer shaft 26. Further, shalt 26 can be insert injection molded onto a secondary shaft 46 that can comprise a polymer extrusion. Secondary shaft 46 can be thermally welded onto a flexible section 30 extruded from a soft polymer, and tip section 32 can be insert injection molded to the flexible section 30. Loading pin 80 can be an extrusion comprising a polymer onto which knob 64 can be insert injection molded.

The retractable pin positioning device 82 can also be configured with a curve 74 (as previously discussed with respect to FIG. 9). In this configuration, the loading pin 80 can comprise flexible materials to enable its retraction around a curve 74 with minimal resistance, such as low-density polyethylene.

Loading pin 80 can also be configured to frictionally retain the folded repair material 40. Referring now to FIG. 16a, a front view of an exemplary friction pin, designated 84 is illustrated. In the illustration, friction pin 84 is shown comprising a “cam-like” cross-sectional geometry and oriented with its large radius 86 concentric with the inside diameter of the tip section 32 and its small radius 88 extending towards the slot 34. Although not shown, it is envisioned the friction pin 84 extends beyond the distal end of the tip section 32. In this configuration, folded repair material 40 can be loaded onto the friction pin 84 and into the slot 34. After loading the folded repair material 40 onto the friction pin 84, the friction pin 84 can be rotated, by rotating knob 64, to compress at least a portion of the folded repair material 40 between the wall of the tip section 32 and the small radius 88 of the friction pin, as illustrated in FIG. 16b. The folded repair material 40 can then be inserted into the anatomy and guided to the defect repair site. Once the folded repair material 40 has be positioned and fixated, the friction pin 84 can be rotated to a position wherein no force is exerted on the folded repair material 40 and then retracted using knob 64, and deployed.

In another embodiment, the loading pin can comprise a non-linear shape to frictionally retain the folded repair material 40. To be more specific, the loading pin can be bowed so that at least a section of the loading pin's length imparts a force on at least a portion of the material 36 against the inside wall of the tip section 32.

The rotatable positioning device 60 can be configured with the features of the retractable pin positioning device 82 and the friction pin 84 feature. This can be achieved by substituting the wire 62 with a flexible torque-coil, tube, or the like, which comprises an internal diameter through which a loading pin 80 can be disposed. Further, knob 64 of the retractable pin device can be disposed, concentrically aligned and adjacent to, the knob 64.

Loading pin 80 can be produced of a flexible material to provide an atraumatic distal end. Also, a separate element can be added to the distal tip of the loading pin 80 to provide such feature. For example, in FIG. 17, an isometric view of an exemplary loading pin 80 is illustrated which comprises an atraumatic pin 90 that was insert injection molded on the distal tip of loading pin 80 utilizing a flexible polymer (e.g., polyurethane). Loading pin can also have a flexible or compressible section located at any point along its length in order to allow deflection and reduce the force imparted upon tissues in the case of user error. The entire distal end of the positioning device can benefit from this feature as well.

Loading pin 80 can comprise additional features. For example, the distal-most end of the loading pin 80 can comprise a light (not shown) for enhanced visualization. This can be achieved by fitting a light, such as a white light emitting diode (LED) on the end of the loading pin and connecting the LED in electrical communication to wires passing through an internal lumen within the loading pin 80. The wires can then be connected in operable communication with a battery disposed within knob 64, which is controlled by a switch disposed on the outside surface of the knob 64. In another embodiment, the end of the loading pin 80 can comprise electrode(s) (not shown) comprising a conductive metal (such as platinum) which can be connected in electrical communication to wires passing through an internal lumen within loading pin 80 and connected to a controller, which is capable of employing the electrode(s) to provide feedback to the operator when the electrode is in contact, or is not in contact: with human tissues (similar to an endophysiological catheter) For example, in one configuration the controller can comprise an optional grounding pad that can be adhered to the patient and when the electrode comes in contact with bodily tissues a circuit can be completed between the contact pad and the electrode that is utilized by the controller to provide feedback to the operator of the contact. The feedback can be a light located on the device's handle 28 or an audible sound.

Loading pin 80 can also be configured with an internal lumen extending from the distal tip of the loading pin 80 through the length of the loading pin 80 and connected to a connector deposed on the knob 64, wherein a vacuum source and/or a fluid source can be connected to the connection to enable the loading pin 80 to aspirate and/or flush the surgical site.

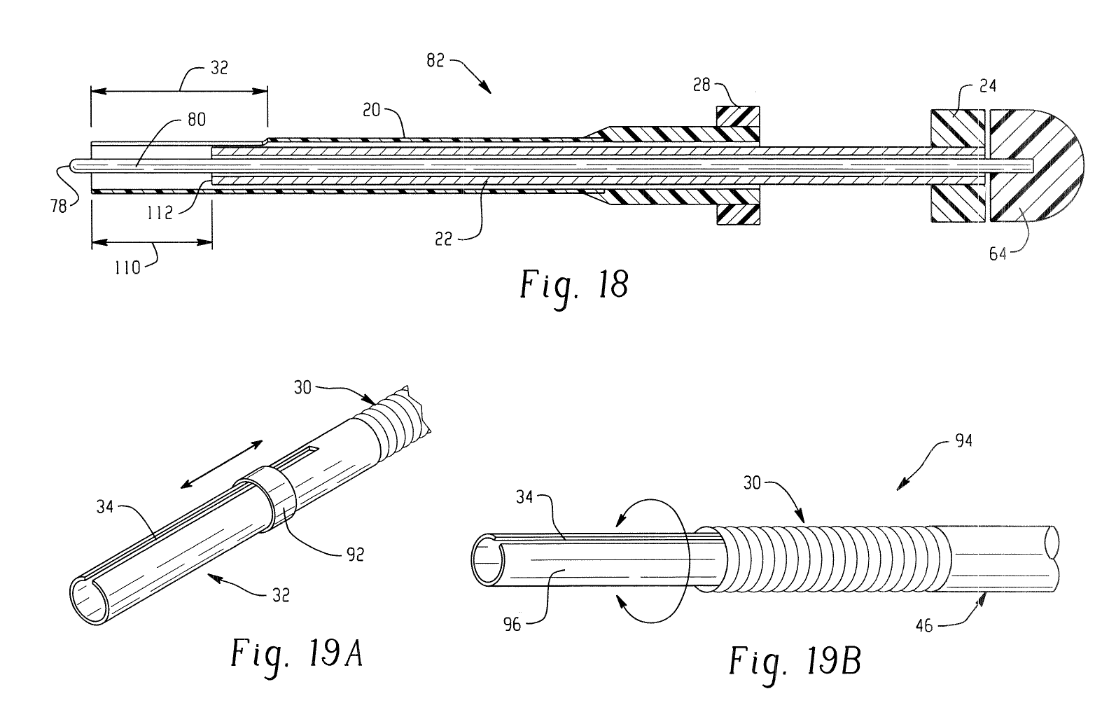

Referring now to FIG. 18, a cross-sectional view of an exemplary retractable pin positioning device 82 configured with a depth stop insert 22 is illustrated. In the illustration, the depth stop insert 22 can be a tubular device that is configured to fit over the loading pin 80 and within the positioning device 20, all are free to translate with respect to one another. The depth stop inhibits the repair material from advancing toward the shaft, away from the distal end of the tip section, e.g., during insertion of the positioning device into the body, through the cannula, and/or through the introducer device. The depth stop can be oriented at a point along the positioning device such that the tip section can receive the repair material, with a side of the repair material located close to the distal end of the tip section (e.g., less than or equal to 2 centimeters (cm), or, more specifically, less than or equal to 1 cm from the end 78 of the tip section), and the material is inhibited from moving toward the shaft, away from the end 78, by, for example, more than 3 cm.

The depth stop insert 22 can comprise an end surface 112 that can function to stop the depth at which a folded repair material 40 can be advanced over loading pin 80. The distance from the distal end of the positioning device 20 to the end surface 112 can be referred to as the useable length 110 of the loading pin 80. In addition, the length of the depth stop insert 22 can be configured for any configuration of folded repair material 40 and/or positioning device 20. It is to be noted that it is desirable that the, distal most end of the folded repair material 40 can be positioned at about the distal most end of the positioning device 20 to minimize the distance required to deploy the folded repair material 40. A collar 24 can be disposed on the proximal end of the depth stop insert 22, between the handle 28 and the knob 64. Depth stop insert can also be useful in aiding the axial release of the material from the positioning device when desired, for example by advancing the stop relative to the end of the positioning device while moving the positioning device in the reverse direction of the material.

The exemplary retractable pin positioning device 82 configured with a depth stop insert 22 can be used to deploy a folded repair material 40 (not shown) by first removing the positioning pin 80 from the depth stop insert 22 by pulling knob 64. Once the loading pin 80 has been removed, the collar 24 can be pushed forward until it contacts the handle 28, during this motion, the end surface 112 advances and pushes the folded repair material 40 out of the distal end 10 of the positioning device 20.

The depth stop insert 22 can be constructed using common methods and materials that can facilitate ease of manufacture and durability, such as a polymer (e.g., polytetrafluorethylene, polyethylene) or metal (e.g., titanium, aluminum, stainless steel), composite, and/or alloy. In one embodiment it is envisioned the depth stop insert 22 is extruded from polyacetals and a polyacetal collar 24 is insert injection molded onto the depth stop insert 22.

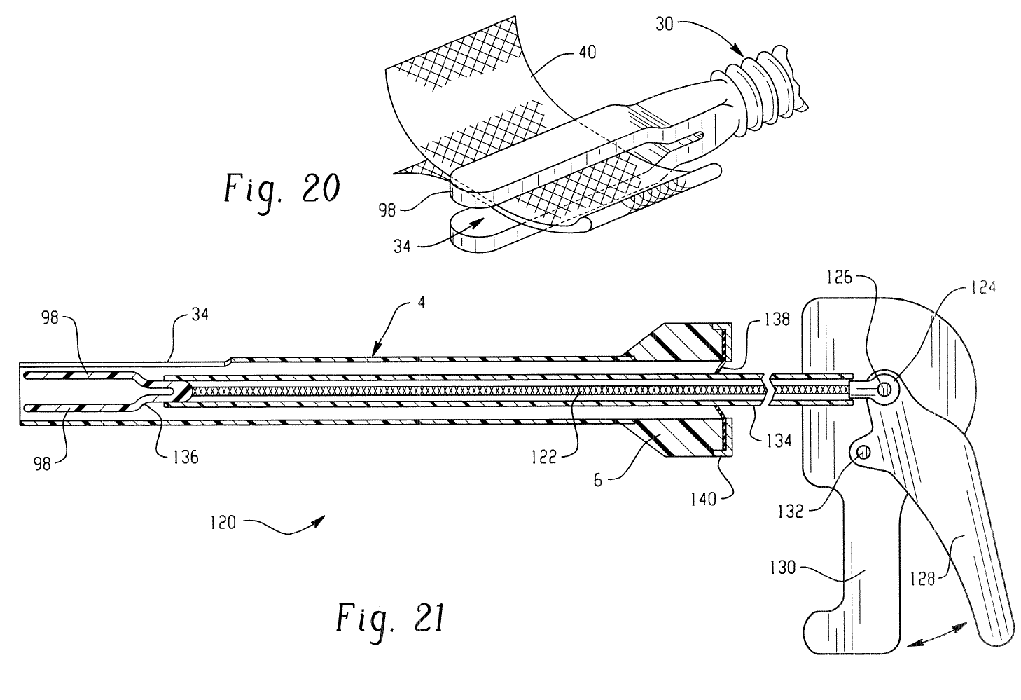

Additional methods of adjusting the slot 34 length of the positioning device 20 are also envisioned. Referring now to FIG. 19a, a partial isometric view of an exemplary slot length adjustment ring, designated 92, is illustrated. In the illustration, a tip section 32 comprising a slot 34 is illustrated with a slot length adjustment ring 92 (hereinafter referred to as ring 92) disposed on the outer surface of the tip section 32. The ring 92 can function to adjust the useable length of the slot 34 by repositioning the ring 92 at various positions along the length of the tip section 32. The useable length of the slot 34 is the length of slot 34 between the ring 92 and the distal end of the tip section 32.

The ring 92 can be adjusted along tip section 32 (as illustrated by the indicating arrow) by overcoming the radially imposed friction imparted by the ring 92 onto the external surface of the tip section 32. Although the ring 92 can comprise any material and any geometry, in one embodiment the ring 92 can comprise a continuous ring of elastomeric material (e.g., polyurethane), which can be stretched slightly around the tip section 32 to impart a radial force and thereby resist movement. In another embodiment, the ring 92 can comprise a non-continuous ring comprising a rigid material (e.g., metal) enabling movement of the ring 92 as the friction imparted on the tip section 32 is overcome with a force acting on the ring 92 in the direction of movement.

Modified rings can be employed as well, wherein a modified ring can be configured to comprise an internal thread that is capable of mating with an external thread integrated into the external geometry of tip section 32. In this configuration the rotation of the modified ring can result in movement of the ring along the length of the tip section 32. Yet further, a modified ring can comprise an internal rib, bulb, cog, or the like, than can be capable of mating with a indentation, groove, pocket, dimple, or the like, disposed in the surface of the tip section 32.

Referring now to FIG. 19b, a partial, isometric view of an exemplary adjustable-tip positioning device, generally designated 94, is illustrated. The adjustable-tip positioning device 94 illustrates an alternative method to adjusting the usable length of slot 34. The device comprises a threaded tip section 96 (threaded section inside flexible section 30, therefore not shown) that can be threaded into flexible section 30 by rotating the tip section, thereby changing the useable length of the slot 34. It is also conceived that the threaded tip section 96 can be threaded into the secondary shaft 46 in a device that does not comprise a flexible section 30. Alternatively, the threaded tip section 96 can be threaded over the outer diameter of the flexible section 30.

Referring now to FIG. 20, a partial, isometric view of an exemplary two-projection tip is depicted, generally designated 98. The two-projection tip 98 comprises two generally elongated elements, wherein each element can be of any cross-sectional geometry (e.g., circular, elliptical, polygonal, irregular, and so forth), and of any individual length. The elements can be disposed to form a gap between the elements that can function as a slot 34.

The two-projection tip 98 functions similarly to a tip section 32 when configured on a positioning device 20 or on a rotatable positioning device 60, or the like. The two-projection tip 98 can be configured so that one or more of its elements can be retractable similar to loading pin 80, to allow the deployment of a material 36 (as disclosed with regard to the retractable pin positioning device 82). One or both elements of the two-projection tip 98 can also be configured with atraumatic tips 90, as well as any of the additional features described for the loading pin 80 (e.g., light, internal lumen for suction or flushing, electrode(s), and so forth). In addition, a slot length adjustment ring 92 can be configured for use with the two-projection tip 98, and/or the two-projection tip 98 can be configured similar to the adjustable-tip positioning device 94, both to enable the adjustment of useable slot 34 length.

Referring now to FIG. 21, a cross-sectional view of an exemplary actuating positioning system 120 is illustrated. The actuating positioning system 120 comprises a two-projection tip 98 that is connected to a cable 122, which is connected to an eyelet 124. The eyelet is connected via a pin 126 to a primary handle 128. Primary handle 128 is mounted within secondary handle 130 via a pivot 132. Connected to secondary handle 130 is a compression tube 134, which is disposed around cable 122 that can freely translate therein. Disposed on the proximal end of the two-projection tip 98 is a tapered feature 136. Moving the primary handle 128 towards the secondary handle 130, in a direction shown by the indicating arrow, can actuate the actuating positioning system 120. The movement of the primary handle pulls cable 122 relative to the compression tube 134, which remains stationary. The cable 122 pulls the tapered feature 136 into the compression tube 134, which causes the two-projections of the two-projection tip 98 to close. This action can be utilized to hold a folded repair material 40 during insertion into the introducer tube 4 and/or manipulate the material 36 once deployed. It is further envisioned a locking mechanism can be incorporated into the handle (such as a releasable ratcheting mechanism) that can temporarily lock the elements in closed position.

In one embodiment, the two-projection tip 98 and cable 122 comprise nickel-titanium alloy and are connected via a weld. Eyelet 124 can comprise a stainless steel and can be crimped onto the proximal end of the cable 122. A stainless steel pin 126 can be inserted through the eyelet 124 to fasten the stainless steel primary handle 128 to the stainless steel secondary handle 130. The pivot 132 can comprise a circular boss on the surface of the primary handle 128 that can be inserted into a mating feature on the secondary handle 130. The secondary handle can be welded to a stainless steel compression tube 134.

Also illustrated in FIG. 21 is a cross-sectional view of the introducer tube 4 (see FIG. 1). Although the introducer tube 4 was previously discussed, FIG. 21 illustrates one embodiment of the gasket 138 that can be employed to create a fluid-tight seal between any device that is capable of passing through the introducer tube 4. The gasket 138 can comprise a polymer (e.g., silicone, polyisoprene) and can be secured within the introducer tube 4 by a cap 140 that can retain the gasket 138 by compression. The cap 140 can be assembled to the element 6 via ultrasonic welding, adhesive bonding, injection molding, or the like.

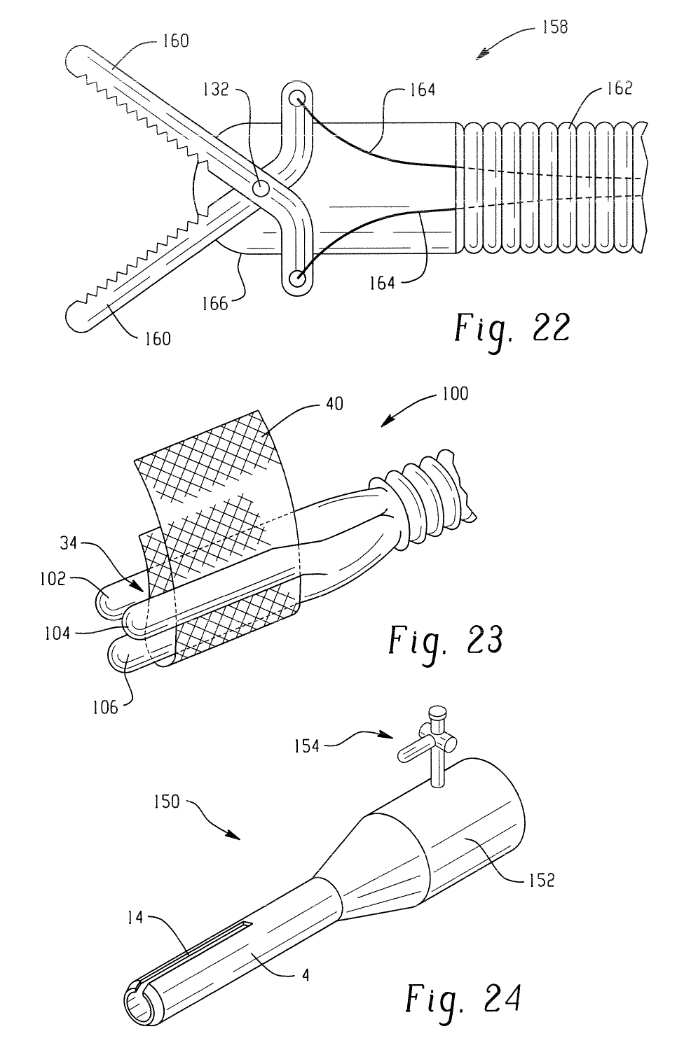

It is to be apparent that linkages and other mechanisms can also be employed for actuating the two-projection tip 98. Refer now to FIG. 22, wherein a partial side view of a modified two element tip, generally designated 158, is illustrated. In the illustration, two jaws 160 are pivotally fastened via a pivot 132 to a support head 166. The jaws 160 can be opened and closed by actuating pull-wires 164, which are free to translate within a coil. The pull-wires 164 can be actuated by hand via a handle (not shown). One exemplary method of manufacturing the modified two-projection tip 158 is by first metal injection molding the jaws 160, which can then be attached to a pivot 132 comprising a stainless steel pin that can be inserted through the support head 166 and welded thereat. The pull-wires 164 can be threaded through holes in the jaws 160 and crimped to the pull-wire 164, forming a loop. The support head 166 can be welded to the coil 162.

The two-projection tip 98, modified two element tip 158, compression tube 134, cable 122, eyelet 124, pin 126, primary handle 128, secondary handle 130., jaws 160, coil 162, support head 166, and pull-wires 164 can be manufactured from polymers (e.g., polyamide, polyacetal), metals (titanium, stainless steels, aluminum), alloys (nickel-titanium), and so forth.

Referring now to FIG. 23, a partial, isometric view of an exemplary three-projection tip configuration is depicted, generally designated 100. The three-projection tip 100 comprises three generally elongated projections; a first projection 102, a second projection 104, and a third projection 106, wherein each element can be of any cross-sectional geometry (e.g., circular, elliptical, polygonal, irregular, and so forth) and comprise any individual length. First projection 102 and second projection 104 can be disposed parallel to one another forming a gap therebetween that can function similar to a slot 34. The third element 106 can be disposed parallel to the first projection 102 and the second projection 104, and comprise a gap between the first projection 102 and second projection 104 and itself. In this configuration, the third projection 106 can function similar to a loading pin 80.

The three-projection tip 100 functions similar to the tip section 32 with a loading pin 80 during use, when configured on a positioning device 20 or on a rotatable positioning device 60. The three-projection tip 100 can be configured with the capability of the third projection 106 capable of retracting to allow the deployment of a material 36 (as disclosed with regard to the retractable pin positioning device 82). Also, one or more of the elements of the three-projection tip 100 can also be configured with atraumatic tips 90 as well as any of the additional features described for the loading pin 80 (e.g., light, internal lumen for suction or flushing, electrode(s), and so forth). In addition, a slot length adjustment ring 92 can be configured for use with the three-projection tip 100, or the three-projection tip 100 can be configured on an adjustable-tip positioning device 94, to enable the adjustment of the useable slot 34 length.

Referring now to FIG. 24, an isometric view of an exemplary introducer/trocar, generally designated 150, is illustrated. The introducer/trocar 150 enables yet another method of gaining access to the abdominal cavity for the deployment of material 36. The introducer trocar 150 comprises an introducer tube 4 and a trocar 152. The trocar 152 can be configured for blunt dissection (as illustrated) or can comprise a sharp and/or cutting tip. The trocar 152 can be capable of dilating through tissue, muscle, and/or fascia. On the proximal section of the trocar 152 is a collar, which can be gripped to remove the trocar 152 from the introducer tube 4 to open the conduit through the device for the insertion of an instrument or device. The introducer tube 4 can also comprise a port/valve 154 that is capable of connecting to a gas supply (not shown) and control the flow of gas and/or liquids therethrough.

The introducer/trocar 150 is utilized to gain access to the abdominal cavity through an incision or puncture. The trocar 152 dilates and/or cuts tissues to enable access. Once access is gained, the trocar can be removed, leaving the introducer tube 4 in place. It is apparent that the introducer tube can be fitted with a gasket 138 (see FIG. 21) to maintain a barrier between the external environment around the patient and the abdominal cavity. The port/valve 154 can be connected to a gas supply that can insufflate the abdomen. It is envisioned the introducer/trocar 150 can replace the use of a cannula 44 in any of the procedures described herein.

Referring now to FIG. 25, a cross-sectional view of an exemplary introducer/trocar 150 comprising a polymer sheath 156 is illustrated in use with a positioning device that has a tip configured for insertion in to the body, and a folded material inserted in the positioning device and positioned in the slot of the trocar cannula. In the illustration, the polymer sheath 156 is attached to the proximal end of the introducer tube 4. The polymer sheath 156 can be configured so that it can be unrolled down the length of the introducer tube 4 to cover the slot 34 after the folded repair material 40 has been rolled onto the positioning device to reduce or eliminate the leakage of insufflation gasses. More specifically, referring to FIG. 26, a side view of an exemplary introducer tube 4 with an unrolled polymer sheath 156 is illustrated. In the illustration, a wrapped repair material 42 is disposed within the introducer tube 4. Over the wrapped repair material 42 and extending down the introducer tube 4 is the unrolled polymer sheath 156. In this embodiment, the introducer tube 4 comprises a slot that extends from within the abdominal cavity to outside the abdominal cavity through the abdominal 48. As illustrated, it is apparent that the unrolled polymer sheath 156 is capable of covering the lot and therefore capable of maintaining insufflation pressure within the abdomen.

The polymer sheath 156 can comprise a polymer (e.g., silicone, polyurethane, latex). In one method of manufacture, the polymer sheath 156 can be formed from a dip-coating process employing latex rubber. The polymer sheath 156 can then be rolled and glued to the introducer tube using an adhesive (e.g., polyurethane, latex). The polymer sheath 156 can comprise a thickness of about 0.004 inches to about 0.025 inches and can be produced with a diameter configured for the outside diameter of the introducer tube 4 employed. The length of the polymer sheath 156 is desirably configured so that in an unrolled configuration the distal end of the sheath does not extend beyond the distal most tip of the introducer tube 4, which would interfere with the deployment of the wrapped repair material 42.

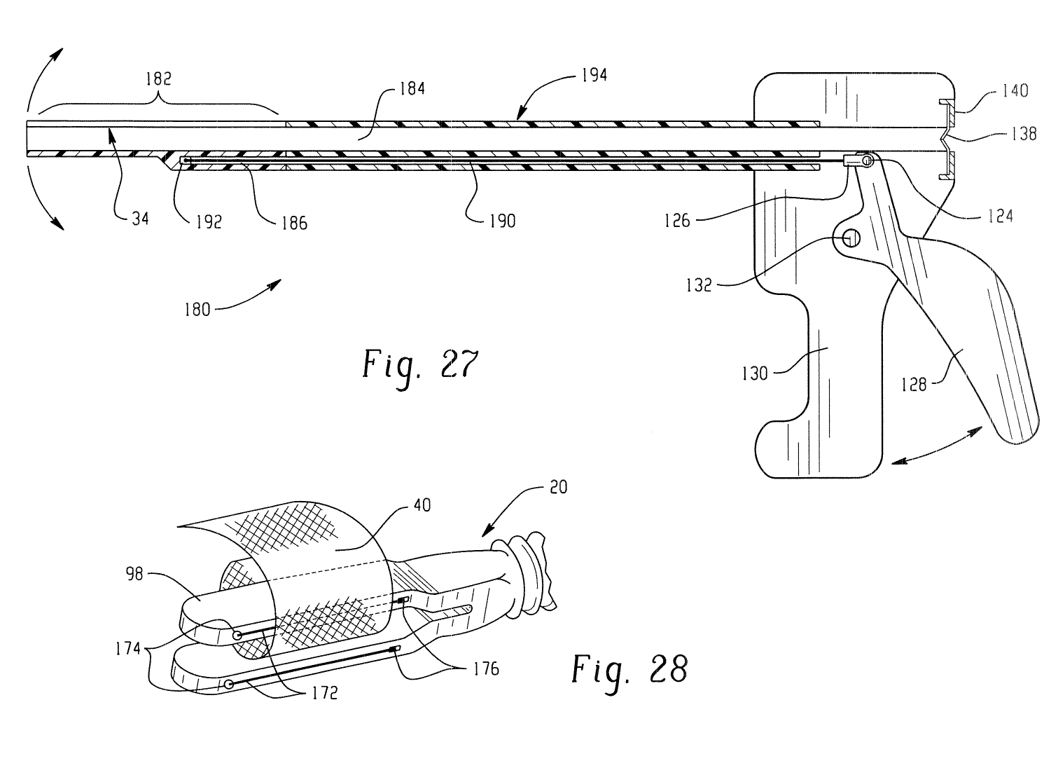

Referring now to FIG. 27, a cross-sectional view of an exemplary steerable positioning device, generally designated 180, is illustrated. In the illustration, the steerable positioning device 180 comprises a deflectable tip section 182, which comprises a slot 34 and an internal lumen 184. A pull-wire lumen 186 is adjacently disposed to the lumen 184. The lumen 184 can accept a loading pin 80 (not shown). Disposed inside the pull-wire lumen 186 is a wire 190 that is connected to an anchor 192 disposed at the distal end of the pull-wire lumen 186. The anchor 192 is attached to the deflectable tip section 182. The deflectable tip section 182 is connected to a multi-lumen tubing 194, which is attached to a secondary handle 130. The wire 190 extends through, and is capable of translating within, the multi-lumen tubing 194, and is connected to an eyelet 126. The eyelet 126 is connected via a pin 126 to a primary handle 128. Primary handle 128 is mounted within secondary handle 130 via a pivot 132. The primary handle 128 can be actuated towards the secondary handle 130, in a direction shown by the indicating arrow. Actuating the primary handle 128 creates a tensile force on the wire 190, which pulls on the anchor 192 causing the deflectable tip section 182 to deflect in the direction as indicated by the indicating arrow.

The steerable positioning device 180 can be loaded with a wrapped repair material 42 that can be positioned around a positioning pin 80 (not shown). The device can be inserted through an introducer tube 4 and advanced toward a defect. The primary handle 128 can be actuated to deflect the deflectable tip section 182 to position a folded repair material 40 in a desired location with respect to the defect. The folded repair material 40 can then be fixedly attached (e.g., staples, anchors, sutures) to the bodily tissues, the loading pin 80 can be retracted, and the steerable positioning device 180 can be retracted to deploy the material 36, which can thereafter be further positioned and secured.

The materials employed for the steerable positioning device can comprise polymers (e.g., acrylonitrile butadiene styrene, polyurethane), metals (e.g., titanium, stainless steel), and alloys (e.g., nickel titanium). To be more specific, the deflectable tip section 182 can be extruded from polyurethane comprising a durometer of about 45 A to about 85 A, which can be thermally bonded to a polyurethane multi-lumen tubing 194 comprising a durometer of about 70 D to about 90 D durometer (A and D indicate durometers measured via the A and D-Scales per ASTM D2240-2005). The multi-lumen tubing 194 can be adhesively bonded to a stainless steel secondary handle 130. A stainless steel primary handle 128 that can be inserted into a mating feature on the secondary handle 130 can comprise a circular boss machined on the surface of the primary handle that can act as a pivot 132. The wire 190 extending through the multi-lumen tubing 194 can be crimped to the eyelet 126 that is connected to the primary handle 128 via a stainless steel pin 124.

Referring now to FIG. 28, an isometric view of an exemplary two-projection tip 98 comprising extension wires 172 is illustrated. In the illustration a two-projection tip 98 is modified with extension wires 172 that are connected to the elements near the distal end of the device and extend along the sides of element along the length of the tip on one or both sides. The extension wires continue through the positioning device 20 to a handle (not shown) that is capable of advancing and retracting the extension wires, which are free to translate within the positioning device 20.

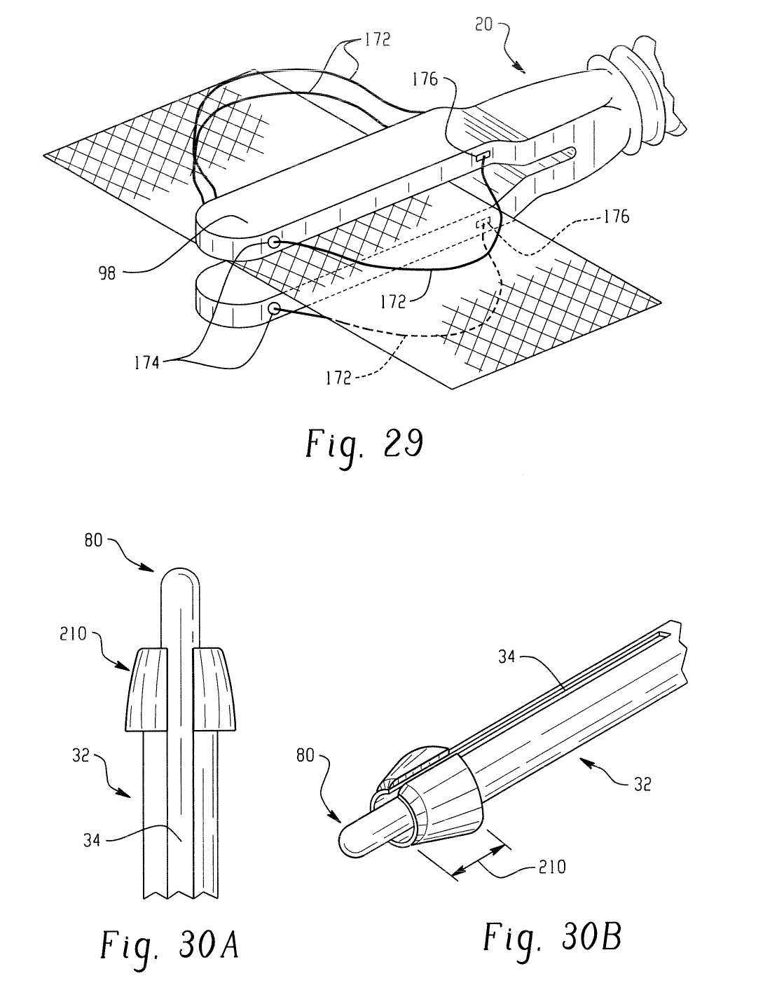

The two-projection tip 98 comprising extension wires 172 comprises a gap formed between the elements wherein material 36 can be disposed. The material 36 can be folded to form a folded repair material 40 and wrapped either by hand/or by employing an introducer tube 4 around the two-projection tip 98 to form a wrapped repair material 42. The wrapped repair material 42 can be inserted into the abdomen utilizing any method disclosed herein. Once the material has been inserted into the abdomen the positioning device 20 can be rotated to unroll the material 36. Once unrolled, the extension wires 172 can be advanced and extended over the material 36, as illustrated in FIG. 29.

The extensions wires 172 can support the material 36 as it is advanced to the defect site and maneuvered thereat to acquire a desirable position prior to securing the material 36 thereat. Once secured, the extension wires 172 can be retracted and the positioning device 20 can be retracted to deploy the material 36 from the two-projection tip 98. The positioning device can thereafter be removed.

In another embodiment, the extension wires 172 can be connected on both ends to the two-projection tip 172. In this configuration, a material 36 can be inserted between the elements and extension wires 172 and the material 36 and wires can be wound around the two-projection tip 98. The wires will resist the wrapping and will biased to return to a non-wrapped configuration. This bias can provide assistance unwrapping the wrapped repair material 42 once inserted into the abdomen. In addition., any tip comprising fixed extension wires 172 will also negate the operation of deploying the extension wires 172 by the operator, and will reduce the complexity of the device.

The components and devices disclosed herein can comprise any material, however polymers, such as, but not limited to, polyetherimide, polysulfone, polypropylene, polycarbonate, polyethylene, polytetrafluorethylene, polyurethane, polystyrene, polyvinylfluoride polyimide, polyamines, and so forth, as well as reaction products, copolymers, mixtures, alloys, and so forth) and metals (e.g., steels, titanium, aluminum, alloys, and so forth) can be employed for ease of manufacturing and biocompatible. Furthermore, one or more coatings can he employed far adding desirable properties to the devices such as but not limited to, lubricity non-conductivity, anti-microbial properties, and so forth.

It is to be apparent that various materials 36 are commercially available and can comprise various geometries, materials, and properties (e.g., TiMESH® available from GfE Gesellschaft für Elektrometallurgie GmbH, Germany, or PROLITE® available from Atrium Medical, Hudson, N.H., Parietex and Parietex Compsotite from Sofradim Corporation, or PROLENE®, or ULTRAPRO® surgical meshes commercially available from Ethicon Inc., Somerville, N.J.). However it is to be apparent that the devices disclosed herein are configurable to function with all repair materials, such as, but not limited to, natural tissues, polymer films, fabrics, and so forth.

The material delivery system and associated devices disclosed herein provide physicians with device systems that can reduce the challenges of introducing and positioning of repair materials prior to fixation. These devices can also potentially reduce procedure times.

Ranges disclosed herein are inclusive and combinable (e.g., ranges of “up to about 25 wt %, or, more specifically, about 5 wt % to about 20 wt %”, is inclusive of the endpoints and all intermediate values of the ranges of “about 5 wt % to about 25 wt %,” etc.). “Combination” is inclusive of blends, mixtures, derivatives, alloys, reaction products, and so forth. Furthermore, the terms “first,” “second,” and so forth, herein do not denote any order, quantity, or importance, but rather are used to distinguish one element from another, and the terms “a” and “an” herein do not denote a limitation of quantity, but rather denote the presence of at least one of the referenced item. The modifier “about” used in connection with a quantity is inclusive of the state value and has the meaning dictated by context, (e.g., includes the degree of error associated with measurement of the particular quantity). The suffix “(s)” as used herein is intended to include both the singular and the plural of the term that it modifies, thereby including one or more of that term (e.g., the colorant(s) includes one or more colorants). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and can or can not be present in other embodiments. In addition, it is to be understood that the described elements can be combined in any suitable manner in the various embodiments.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

While the disclosure has been described with reference to an exemplary embodiment, it will be understood by those skilled in the art that various changes can be made and equivalents can be substituted for elements thereof without departing from the scope of the disclosure. In addition, many modifications can be made to adapt a particular situation or material to the teachings of the disclosure without departing from the essential scope thereof. Therefore, it is intended that the disclosure not be limited to the particular embodiment disclosed as the best mode contemplated for carrying out this disclosure, but that the disclosure will include all embodiments falling within the scope of the appended claims.

What is claimed is:

1. A repair system, comprising:
a positioning device comprises a shaft, a tip section, and a depth stop;
wherein the tip section comprises a material receiving area that is configured to receive a fold of a folded material;
wherein the tip section is configured to enable wrapping of the folded material around the tip section; and
wherein the depth stop is configured to inhibit the folded material from moving toward the shaft beyond a desired point.

2. The system of claim 1, further comprising an introducer device comprising an element having a tapered inner diameter that tapers toward a distal end, and wherein a largest outer diameter of at least the tip section is less than a smallest inner diameter of the element.

3. The system of claim 1, wherein the element further comprises a first valve.

4. The system of claim 1, wherein the introducer device further comprises a tube that extends from the element, wherein the tube is configured to receive the tip section.

5. The system of claim 1, wherein the positioning device further comprises a flexible section located proximate the tip section.

6. The system of claim 1, wherein the material receiving area is selected from the group consisting of a slot, projections,jaws, and combinations comprising at least one of the foregoing.

7. The system of claim 1, wherein the positioning device further comprises a loading pin located in the tip section and configured to accept the folded material.

8. The system of claim 7, wherein the loading pin is selected from the group consisting of a friction pin, a retractable pin, atraumatic pin, rotatable pin, and combinations comprising at least one of the foregoing pins.

9. The system of claim 1, wherein the positioning device further comprises a wire that extends from the tip section, through the shaft, to a first knob, wherein the knob is capable of controlling the movement of the tip section.

10. The system of claim 1, wherein positioning device further comprises a handle having an axis, and wherein the tip section can be oriented on a different axis than the handle.

11. The system of claim 1, wherein positioning device further comprises a handle and wherein the shaft further comprises a joint in operational communication with the handle via a wire.

12. The system of claim 1, wherein the tip section is rotatable.

13. The system of claim 1, further comprising an introducer device comprising a tube with a second material receiving area, wherein the positioning device has a size and geometry to extend through the tube such that at least a portion of the tip section can protrude from a distal end of the introducer device, and wherein the second material receiving area enables the introduction of the fold through the distal end and enables the folded material to extend through the second material receiving area before being wound around the tip section.

14. The system of claim 1, wherein the tip section comprises a distal end opposite the shaft, and wherein the distal end comprises a taper from the distal end into the material receiving area.

15. The system of claim 1, wherein the positioning device has an adjustable length.

16. The system of claim 1, wherein the shaft comprises a tapered section that becomes gradually narrower toward the tip section.

17. The system of claim 1, wherein the tip section farther comprises an extension wire, and wherein the extension wire is configured to extend from the tip section to support the folded material.

18. The system of claim 1, further comprising a visual indicator configured to indicate the orientation of the tip section.

19. The system of claim 1, further comprising a rotation mechanism in operable communication with the tip section and configured to rotate the tip section.

20. The system of claim 1, further comprising a flexible support member disposed on the tip section and configured to contact the folded material.

21. A repair system, comprising:
a positioning device comprising a shaft and a tip section,
wherein the tip section comprises a material receiving area and a retractable extension wire;
wherein the tip section is configured to receive material and retain the material until it is deployed at a defect site, and
wherein the extension wire is configured to extend from the tip section to support the material during deployment.

22. A method for operating a repair system, comprising:
folding a material to form a folded material;
inserting the folded material into a material receiving area in a positioning device, wherein the positioning device comprises shaft and a tip section, wherein the tip section comprises the material receiving area;
inserting the tip section and folded material into a tapered element at a proximal end of an introducer device, wherein the introducer device comprises the proximal end for receiving the tip section and a distal end for deploying the material;
wrapping the folded material around the tip section to form a wrapped material;
passing the wrapped material through the introducer device;
unwrapping the wrapped material to form an unwrapped material;
positioning the unwrapped material in a desired location; and
securing the unwrapped material to a repair site.

23. The method of claim 22, wherein the introducer device extends into a body to near a repair site.

24. The method of claim 22, wherein, after wrapping the folded material, further comprising introducing the introducer device to a body such that the distal end is disposed near a repair site.

25. The method of claim 24, wherein introducing the introducer device to a body further comprises pass a cannula through an abdominal wall such that the cannula extends from outside the body into an abdominal cavity, and passing the introducer device through a cannula.

26. The method of claim 22, wherein wrapping the folded material comprising rotating the tip section as it passes through the tapered element, and further comprising after passing the wrapped material through the introducer device, passing the wrapped material through a cannula into a body.

27. The method of claim 22, wherein wrapping the folded material around the tip section further comprising mechanically rotating the tip section.

28. The method of claim 22, further comprising, subsequent to unwrapping the wrapped material, supporting the unwrapped material with extension wires.

29. The method of claim 22, wherein inserting the folded material into a material receiving area further comprises disposing the folded material around a loading pin.

30. The method of claim 22, wherein the positioning device further comprises a flexible section located proximate the tip section, and wherein positioning the unwrapped material further comprises bending the flexible section.

31. A method for operating a repair system, comprising:
folding a material to form a folded material;
inserting the folding material receiving area in a positioning device, wherein the positioning device comprises shaft and a tip section, wherein the tip section comprises the material receiving area and a conical tip;
wrapping the folded material around the tip section, adjacent to a wide end of the conical tip to form a wrapped material;
passing the wrapped material through an abdominal wall into an abdominal cavity;
unwrapping the wrapped material to form an unwrapped material;
positioning the unwrapped material in a desired location; and
securing the unwrapped material to a repair site.

32. The method of claim 32, further comprising, subsequent to unwrapping the wrapped material, supporting the unwrapped material with extension wires.

33. A method for operating a repair system, comprising:
folding a material to form a folded material;
insert a positioning device through an introducer device such that a tip section of the positioning device extends out of a distal end of the introducer device, wherein the tip section comprises a first material receiving area;
inserting the folded material into the first material receiving area, wherein the folded material can extend into the first material receiving area up to a stop;
inserting the tip section and folded material into a second material receiving area;
wrapping the folded material around the tip section to form a wrapped material;
introducing the wrapped material to an abdominal cavity;
unwrapping the wrapped material to form an unwrapped material;
positioning the unwrapped material in a desired location; and
securing the unwrapped material to a repair site.
